SEQUENCE LISTING (6 sequences)

SEQ ID NO: 1 - DNA, 1783 bp, Homo sapiens
tcgagcccgctttccagggaccctacctgagggcccacaggtgaggcagcctggcctagcaggccccacgccaccgcctctgcctccaggccgcccgctgctgcggggccaccatgctcctgcccaggcctggagactgacccgaccccggcactacctcgaggctccgcccccacctgctggaccccagggtcccaccctggcccaggaggtcagccagggaatcattaacaagaggcagtgacatggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctgaccggtggcttctcgctgcgcagcctccagggcccgaggtgatcccggtggtgggatccacgctgggccgaggatgggacgtttttcttcttgggcccggtccacaggtccaaggacaccctccctccagggtcctctcttccacagtggcgggcccactcagccccgagaccacccaacctcaccctcctgacccccatgtaaatattgttctgctgtctgggactcctgtctaggtgcccctgatgatgggatgctctttaaataataaagatggttttgatt

SEQ ID NO: 2 - protein, 417 aa, Homo sapiens
MAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 3 - DNA, 18 bp, Homo sapiens
cactcagccccgagacca

SEQ ID NO: 4 - DNA, 25 bp, Homo sapiens
agtcccagacagcagaacaatattt

SEQ ID NO: 5 - DNA, 23 bp, Homo sapiens
ccaacctcaccctcctgaccccc

SEQ ID NO: 6 - protein, 19 aa, Homo sapiens
WIFQAIKTHSEASGMVTQL

(19) United States
(12) Patent Application Publication
(10) Pub. No.: US 2003/0049645 A1
(43) Pub. Date: Mar. 13, 2003

(54) Amplified cancer gene hepsin

(76) Inventors: David Mu, Jericho, NY (US); Scott Powers, Greenlawn, NY (US)

Correspondence Address: HELLER EHRMAN WHITE & MCAULIFFE, Suite 300, 1666 K Street, N.W., Washington, DC, 20006, US

(21) Appl. No.: 10/073,060

(22) Filed: Feb. 12, 2002

Related U.S. Application Data
(60) Provisional application No. 60/268,361, filed on Feb. 14, 2001.

(51) Int. Cl. C12Q 1/68; G06F 19/00; C12P 19/34

(52) U.S. Cl. 435/6; 435/91.2; 702/20

(57) ABSTRACT

There are disclosed methods and compositions for the diagnosis, prevention, and treatment of tumors and cancers in mammals, for example, humans, utilizing the hepsin gene, which are amplified ovarian, and/or prostate, and/or breast, and/or lung cancer genes. The hepsin gene, its expressed protein products and antibodies are used diagnostically or as targets for cancer therapy; they are also used to identify compounds and reagents useful in cancer diagnosis, prevention, and therapy.

This application relates to U.S. Ser. No. 60/268,361, filed Feb. 14, 2001, the entirety of which is hereby incorporated by reference.

### BACKGROUND OF THE INVENTION

1. Field of the Invention

The present invention relates to oncogenes and to cancer diagnostics and therapeutics. More specifically, the present invention relates an amplified and overexpressed hepsin gene is involved in certain types of cancers. The invention pertains to the amplified gene, its encoded proteins, and antibodies, inhibitors, activators and the like in cancer screening and anti-cancer therapy, including ovarian cancer and prostate cancer.

2. Background of the Invention

Cancer is the second leading cause of death in the United States, after heart disease (Boring, et al., CA Cancer J. Clin., 43:7, 1993), and it develops in one in three Americans. One of every four Americans dies of cancer. Cancer features uncontrolled cellular growth, which results either in local invasion of normal tissue or systemic spread of the abnormal growth known as metastasis. A particular type of cancer or a particular stage of cancer development may involve both elements.

The division or growth of cells in various tissues functioning in a living body normally takes place in an orderly and controlled manner. This is enabled by a delicate growth control mechanism, which involves, among other things, contact, signaling, and other communication between neighboring cells. Growth signals, stimulatory or inhibitory, are routinely exchanged between cells in a functioning tissue. Cells normally do not divide in the absence of stimulatory signals, and will cease dividing when dominated by inhibitory signals. However, such signaling or communication becomes defective or completely breaks down in cancer cells. As a result, the cells continue to divide; they invade adjacent structures, break away from the original tumor mass, and establish new growth in other parts of the body. The latter progression to malignancy is referred to as “metastasis.”

Cancer generally refers to malignant tumors, rather than benign tumors. Benign tumor cells are similar to normal, surrounding cells. These types of tumors are almost always encapsulated in a fibrous capsule and do not have the potential to metastasize to other parts of the body. These tumors affect local organs but do not destroy them; they usually remain small without producing symptoms for many years. Treatment becomes necessary only when the tumors grow large enough to interfere with other organs. Malignant tumors, by contrast, grow faster than benign tumors; they penetrate and destroy local tissues. Some malignant tumors may spread throughout the body via blood or the lymphatic system. The unpredictable and uncontrolled growth makes malignant cancers dangerous, and fatal in many cases. These tumors are not morphologically typical of the original tissue and are not encapsulated. Malignant tumors commonly recur after surgical removal.

Treatment, therefore, ordinarily targets malignant cancers or malignant tumors. The intervention of malignant growth is most effective at the early stage of the cancer development. It is thus exceedingly important to discover sensitive markers for early signs of cancer formation and to identify potent growth suppression agents associated therewith. The invention of such diagnostic and treatment agents hinges upon the understanding of the genetic control mechanisms for cell division and differentiation, particularly in connection to tumorigenesis. Cancer is caused by inherited or acquired mutations in cancer genes, which have normal cellular functions and which induce or otherwise contribute to cancer once mutated or expressed at an abnormal level. Certain well-studied tumors carry several different independently mutated genes, including activated oncogenes and inactivated tumor suppressor genes. Each of these mutations appears to be responsible for imparting some of the traits that, in aggregate, represent the full neoplastic phenotype (Land et al., Science, 222:771, 1983; Ruley, Nature, 4:602, 1983; Hunter, Cell, 64:249, 1991).

One such mutation is gene amplification. Gene amplification involves a chromosomal region bearing specific genes undergoing a relative increase in DNA copy number, thereby increasing the copies of any genes that are present. In general, gene amplification results in increased levels of transcription and translation, producing higher amounts of the corresponding gene mRNA and protein. Amplification of genes causes deleterious effects, which contribute to cancer formation and proliferation (Lengauer et al. Nature, 396:643-649 (1999)).

It is commonly appreciated by cancer researchers that whole collections of genes are demonstrably overexpressed or differentially expressed in a variety of different types of tumor cells. Yet, only a very small number of these overexpressed genes are likely to be causally involved in the cancer phenotype. The remaining overexpressed genes likely are secondary consequences of more basic primary events, for example, overexpression of a cluster of genes, involved in DNA replication. On the other hand, gene amplification is established as an important genetic alteration in solid tumors (Knuutila et al., Am J Pathol 1998 152(5):1107-23; Knuutila et al., Cancer Genet Cytogenet. 0:2-(1998)).

The overexpression of certain well known genes, for example, c-myc, have been observed at fairly high levels in the absence of gene amplification (Yoshimoto et al, 1986, JPN J Cancer Res, 77(6):540-5), although these genes are frequently amplified (Knuutila et al., Am J Pathol 1998 152(5):1107-23) and thereby activated. Such a characteristic is considered a hallmark of oncogenes. Overexpression in the absence of amplification may be caused by higher transcription efficiency in those situations. In the case of c-myc, for example, Yoshimoto et al. showed that its transcriptional rate was greatly increased in the tested tumor cell lines. The characteristics and interplay of overexpression and amplification of a gene in cancer tissues, therefore, provide significant indications of the gene's role in cancer development. That is, increased DNA copies of certain genes in tumors, along with and beyond its overexpression, may point to their functions in tumor formation and progression.

Thus, the invention, as well characterization of amplified cancer genes, in general, along with and in addition to their features of overexpression or differential expression, will be a promising avenue that leads to novel targets for diagnostic and therapeutic applications in cancer.

Additionally, the completion of the working drafts of the human genome and the paralleled advances in genomics technologies offer new promises in the identification of effective cancer markers and the anti-cancer agents. The high-throughput microarray detection and screening technology, computer-empowered genetics and genomics analysis tools, and multi-platform functional genomics and proteomics validation systems, all lend themselves in applications in cancer research and findings.

With the advent of modem sequencing technologies and genomic analyses, many unknown genes and genes with unknown or partially known functions are revealed.

Hepsin is a trypsin-like serine protease; its gene was first cloned in 1988 by Leytus et al. from human liver and hepatoma cell line mRNAs (Biochemistry 1988, 27(3):1067-74). The hepsin cDNA is approximately 1.8 kb in length with a coding region of 1251 nucleotides, which encodes a protein of 417 amino acids. The amino acid sequence encoded by the cDNA for hepsin shows a high degree of identity to pancreatic trypsin and other serine proteases. It also contains a cleavage site for protease activation and a highly conserved region surrounding the His-Asp-Ser catalytic center; thus, it resembles zymogens of serine proteases. Leytus et al. also identified a putative transmembrane domain in the coding sequence, which may serve to anchor hepsin to the cell membrane in such a manner that its catalytic domain is extracellular.

The activity of hepsin as an extracellular protease implicates a potential role in tumor progression. Extracellular proteases mediate the digestion of neighboring extracellular matrix components in initial tumor growth, allow shedding or desquamation of tumor cells into the surrounding environment, provide the basis for invasion of basement membranes in target metastatic organs, and are required for release and activation of many growth and angiogenic factors. The overexpression of the hepsin gene was first reported by Tanimoto et al in 1997 (Cancer Res 1997, 57(14):2884-7). Tanimoto et al. determined the level of expression of the hepsin gene in ovarian carcinomas and ovarian tumors compared to normal ovarian tissue, and found that hepsin is frequently overexpressed in ovarian tumors. No hepsin expression was found in normal adult tissue, other than a low level of expression in prostate. Tanimoto et al. stated that the role of hepsin in tumor cell growth and spread is “unclear” but speculated that it may contribute to the invasive nature or growth capacity of ovarian tumors. Tanimoto et al. further speculated that ovarian tumor growth and spread required coordination of a matrix of different protease activities and that this “may” offer an opportunity to use expression of the matrix as a potential diagnostic indicator or as a atarget for therapy. Notably, Tanimoto et al. did not describe any evidence that: (i) the hepsin gene is amplified in tumor tissue; (ii) that hepsin is overexpressed in tumors of any tissue other than ovary, (iii) hepsin may be directly implicated in ovarian tumorigenesis and cancer progression or (iv) that hepsin alone may provide opportunities for diagnostic and therapeutic utilities.

It is apparent, therefore, that identification of amplified and/or overexpressed genes, including oncogenes, that are involved in tumorigenesis and cancer progression are desired. It is also apparent that methods of using these genes in cancer diagnosis and treatment are highly desirable. The technologies and knowledge thus call for the invention of novel targets for the diagnostic markers involved in tumorigenesis and new potent anticancer treatment regimen.

### SUMMARY OF THE INVENTION

The present invention relates to isolation, characterization, overexpression and implication of genes, including amplified genes, in cancers, methods and compositions for the diagnosis, prevention, and treatment of tumors and cancers, for example, ovarian cancer, in mammals, for example, humans. The invention is based on the finding of novel traits of a gene, hepsin, which is originally identified as a gene encoding trypsin-like serine protease.

Hepsin gene encodes serine protease, which is expressed in human tumors. As disclosed herein, hepsin gene appears to be at the epicenter of amplification region in quantitative PCR analysis of human malignant tumors, for example, ovarian cancer. As disclosed for the first time, hepsin gene is amplified and overexpressed in human ovarian tumor samples, for example.

These novel traits include the overexpression of the hepsin gene in certain cancers, for example, ovarian cancer, prostate cancer, lung cancer, or breast cancer, etc., and the frequent amplification of hepsin DNA in cancer cells. The hepsin gene and its expressed protein product can thus be used diagnostically or as targets for cancer therapy; and they can also be used to identify and design compounds useful in the diagnosis, prevention, and therapy of tumors and cancers (for example, ovarian cancer, prostate cancer, lung cancer, or breast cancer, etc.).

According to one aspect of the present invention, the use of hepsin in gene therapy, development of antisense nucleic acids and small interfering RNAs (siRNAs), and development of immunodiagnostics or immunotherapy are provided. The present invention also includes production and the use of antibodies, for example, monoclonal, polyclonal, single-chain and engineered antibodies (including humanized antibodies) and fragments, which specifically bind hepsin proteins and polypeptides. The invention also features antagonists and inhibitors of hepsin proteins that can inhibit one or more of the functions or activities of hepsin proteins. Suitable antagonists can include small molecules (molecular weight below about 500), large molecules (molecular weight above about 500), antibodies, including fragments and single chain antibodies, that bind and “neutralize” hepsin proteins, polypeptides and which compete with a native form of hepsin proteins for binding to a protein which may naturally interact with hepsin proteins for the latter's function, and nucleic acid molecules that interfere with transcription of the hepsin genes (for example, antisense nucleic acid molecules, ribozymes and small interfering RNAs (siRNAs). Useful agonists, ones that may induce certain mutants of hepsin thereby attenuating activities of hepsin, also include small and large molecules, and antibodies other than “neutralizing” antibodies.

The present invention further features molecules that can decrease the expression of hepsin by affecting transcription or translation. Small molecules (molecular weight below about 500), large molecules (molecular weight above about 500), and nucleic acid molecules, for example, ribozymes, siRNAs and antisense molecules may all be utilized to inhibit the expression or amplification.

As mentioned above, the hepsin gene sequence also can be employed in an RNA interference context. The phenomenon of RNA interference is described and discussed in Bass, Nature 411: 428-29 (2001); Elbahir et al., Nature 411: 494-98 (2001); and Fire et al., Nature 391: 806-11 (1998), where methods of making interfering RNA also are discussed.

In one aspect, the present invention provides a method for diagnosing a cancer, for example, an ovarian cancer, a prostate cancer, a lung cancer, or a breast cancer, etc., in a mammal, which comprises, for example, obtaining a biological test sample from a region in the tissue that is suspected to be precancerous or cancerous; and measuring in the biological subject the number of hepsin gene copies thereby determining whether the hepsin gene is amplified in the biological test subject, wherein amplification of the hepsin gene indicates a cancer in the tissue.

In another aspect, the present invention provides a method for diagnosing a cancer, for example, an ovarian cancer, a prostate cancer, a lung cancer, or a breast cancer, etc., in a mammal, which comprises, for example, obtaining a biological test sample from a region in the tissue that is suspected to be precancerous or cancerous; obtaining a biological control sample from a region in the tissue or other tissues in the mammal that is normal; and detecting in both the biological test sample and the biological control sample the level of hepsin messenger RNA transcripts, wherein a level of the transcripts higher in the biological subject than that in the biological control sample indicates a cancer in the tissue. In another aspect the biological control sample may be obtained from a different individual or be a normalized value based on baseline values found in a population.

In another aspect, the present invention provides a method for diagnosing a cancer, for example, an ovarian cancer, a prostate cancer, a lung cancer, or a breast cancer, etc., in a mammal, which comprises, for example, obtaining a biological test sample from a region in the tissue that is suspected to be precancerous or cancerous; and detecting in the biological subject the number of hepsin DNA copies thereby determining whether the hepsin gene is amplified in the biological test subject, wherein amplification of the hepsin gene indicates a cancer in the tissue.

Another aspect of the present invention provides a method for diagnosing a cancer, for example, an ovarian cancer, a prostate cancer, a lung cancer, or a breast cancer, etc., in a mammal, which comprises, for example, obtaining a biological test sample from a region in the tissue that is suspected to be precancerous or cancerous; contacting the samples with anti-hepsin antibodies, and detecting in the biological subject the level of hepsin protein expression, wherein a level of the hepsin protein expression higher in the biological subject than that in the biological control sample indicates a cancer in the tissue. In an alternative aspect the biological control sample may be obtained from a different individual or be a normalized value based on baseline values found in a population.

In another aspect, the present invention relates to methods for comparing and compiling data wherein the data is stored in electronic or paper format. Electronic format can be selected from the group consisting of electronic mail, disk, compact disk (CD), digital versatile disk (DVD), memory card, memory chip, ROM or RAM, magnetic optical disk, tape, video, video clip, microfilm, internet, shared network, shared server and the like; wherein data is displayed, transmitted or analyzed via electronic transmission, video display, telecommunication, or by using any of the above stored formats; wherein data is compared and compiled at the site of sampling specimens or at a location where the data is transported following a process as described above.

In another aspect, the present invention provides a method for preventing, controlling, or suppressing cancer growth in a mammalian organ and tissue, for example, in the ovary, prostate, lung, or breast, which comprises administering an inhibitor of hepsin protein to the organ or tissue, thereby inhibiting hepsin protein activities. Such inhibitors may be, inter alia, an antibody to hepsin protein or polypeptide portions thereof, an antagonist to hepsin protein, or other small molecules.

In a further aspect, the present invention provides a method for preventing, controlling, or suppressing cancer growth in a mammalian organ and tissue, for example, in the ovary, prostate, lung, or breast, which comprises administering to the organ or tissue a nucleotide molecule that is capable of interacting with hepsin DNA or RNA and thereby blocking or interfering the hepsin gene functions, respectively. Such nucleotide molecule can be an antisense nucleotide of the hepsin gene, a ribozyme of hepsin RNA; a small interfering RNA (siRNA) or it may be capable of forming a triple helix with the hepsin gene.

In still a further aspect, the present invention provides a method for monitoring the efficacy of a therapeutic treatment regimen for treating a cancer, for example, an ovarian cancer, a prostate cancer, a lung cancer, or a breast cancer, etc., in a patient, for example, in a clinical trial, which comprises obtaining a first sample of cancer cells from the patient; administering the treatment regimen to the patient; obtaining a second sample of cancer cells from the patient after a time period; and detecting in both the first and the second samples the level of hepsin messenger RNA transcripts, wherein a level of the transcripts lower in the second sample than that in the first sample indicates that the treatment regimen is effective to the patient.

In another aspect, the present invention provides a method for monitoring the efficacy of a compound to suppress a cancer, for example, an ovarian cancer, a prostate cancer, a lung cancer, or a breast cancer, etc., in a patient, for example, in a clinical trial, which comprises obtaining a first sample of cancer cells from the patient; administering the treatment regimen to the patient; obtaining the second sample of cancer cells from the patient after a time period; and detecting in both the first and the second samples the level of hepsin messenger RNA transcripts, wherein a level of the transcripts lower in the second sample than that in the first sample indicates that the compound is effective to suppress such a cancer.

In another aspect, the present invention provides a method for monitoring the efficacy of a therapeutic treatment regimen for treating a cancer, for example, an ovarian cancer, a prostate cancer, a lung cancer, or a breast cancer, etc., in a patient, for example, in a clinical trial, which comprises obtaining a first sample of cancer cells from the patient; administering the treatment regimen to the patient; obtaining a second sample of cancer cells from the patient after a time period; and detecting in both the first and the second samples the number of hepsin DNA copies, thereby determining the overall or average hepsin gene amplification state in the first and second samples, wherein a lower number of hepsin DNA copies in the second sample than that in the first sample indicates that the treatment regimen is effective.

In yet another aspect, the present invention provides a method for monitoring the efficacy of a therapeutic treatment regimen for treating a cancer, for example, an ovarian cancer, a prostate cancer, a lung cancer, or a breast cancer, etc., in a patient, which comprises obtaining a first sample of cancer cells from the patient; administering the treatment regimen to the patient; obtaining a second sample of cancer cells from the patient after a time period; contacting the samples with anti-hepsin antibodies, and detecting in the level of hepsin protein expression, in both the first and the second samples. A lower level of the hepsin protein expression in the second sample than that in the first sample indicates that the treatment regimen is effective to the patient.

In still another aspect, the present invention provides a method for monitoring the efficacy of a compound to suppress a cancer, for example, an ovarian cancer, a prostate cancer, a lung cancer, or a breast cancer, etc., in a patient, for example, in a clinical trial, which comprises obtaining a first sample of cancer cells from the patient; administering the treatment regimen to the patient; obtaining a second sample of cancer cells from the patient after a time period; and detecting in both the first and the second samples the number of hepsin DNA copies, thereby determining the hepsin gene amplification state in the first and second samples, wherein a lower number of hepsin DNA copies in the second sample than that in the first sample indicates that the compound is effective.

One aspect of the invention is to provide an isolated hepsin gene amplicon for diagnosing cancer and/or monitoring the efficacy of a cancer therapy, which comprises, for example, obtaining a biological test sample from a region in the tissue that is suspected to be precancerous or cancerous; obtaining a biological control sample from a region in the tissue or other tissues in the mammal that is normal; and detecting in both the biological test sample and the biological control sample the level of hepsin gene amplicon, wherein a level of the amplicon higher in the biological subject than that in the biological control sample indicates a precancerous or cancer condition in the tissue. In an aspect, the biological control sample may be obtained from a different individual or be a normalized value based on baseline values found in a population.

Another aspect of the invention is to provide an isolated hepsin gene amplicon, wherein the amplicon comprises a completely or partially amplified product of hepsin gene, including a polynucleotide having at least about 90% sequence identity to hepsin gene, for example, SEQ ID NO: 1, a polynucleotide encoding the polypeptide set forth in SEQ ID NO: 2, or a polynucleotide that is overexpressed in tumor cells having at least about 90% sequence identity to the polynucleotide of SEQ ID NO: 1 or the polynucleotide encoding the polypeptide set forth in SEQ ID NO: 2.

In yet another aspect, the present invention provides a method for modulating hepsin activities by contacting a biological subject from a region that is suspected to be precancerous or cancerous with a modulator of the hepsin protein, wherein the modulator is, for example, a small molecule.

In still another aspect, the present invention provides a method for modulating hepsin activities by contacting a biological subject from a region that is suspected to be precancerous or cancerous with a modulator of the hepsin protein, wherein said modulator partially or completely inhibits transcription of hepsin.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. All publications, patent applications, patents, database records, for example, those in SWISS-PROT, GENBANK, EMBL, etc., and other references and citations mentioned herein are incorporated by reference in their entirety. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, that the detailed description and the specific examples, while indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Figure shows the epicenter mapping of human chromosome region 19q13 amplicon which includes hepsin locus. The number of DNA copies for each sample is plotted on the Y-axis, and the X-axis corresponds to nucleotide position based on Human Genome Project working draft sequence (htt://genome.ucsc.edu/goldenPath/aug2001Tracks.html).

FIG. 2: Figure shows differential sensitivity of ovarian cancer cells to hepsin antibodies.

### DETAILED DESCRIPTION OF THE INVENTION

The present invention provides methods and compositions for the diagnosis, prevention, and treatment of tumors and cancers, for example, an ovarian cancer, a prostate cancer, a lung cancer, or a breast cancer, etc., in mammals, for example, humans. The invention is based on the findings of novel traits of the hepsin gene that encodes a serine protease in cancer cells. The hepsin genes and their expressed protein products can thus be used diagnostically or as targets for therapy; and, they can also be used to identify compounds useful in the diagnosis, prevention, and therapy of tumors and cancers (for example, ovarian cancer, prostate cancer, lung cancer, or breast cancer, etc.).

The present invention, for the first time, provides an isolated amplified hepsin gene. This invention also provides that the hepsin gene is frequently amplified and overexpressed in tumor cells, for example, human ovary, prostate, lung, or breast tumors.

Definitions:

A “cancer” in an animal refers to the presence of cells possessing characteristics typical of cancer-causing cells, for example, uncontrolled proliferation, loss of specialized functions, immortality, significant metastatic potential, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells.

The phrase “detecting a cancer” or “diagnosing a cancer” refers to determining the presence or absence of cancer or a precancerous condition in an animal. “Detecting a cancer” also can refer to obtaining indirect evidence regarding the likelihood of the presence of precancerous or cancerous cells in the animal or assessing the predisposition of a patient to the development of a cancer. Detecting a cancer can be accomplished using the methods of this invention alone, in combination with other methods, or in light of other information regarding the state of health of the animal.

A “tumor,” as used herein, refers to all neoplastic cell growth and proliferation, whether malignant or benign, and all precancerous and cancerous cells and tissues.

The term “precancerous” refers to cells or tissues having characteristics relating to changes that may lead to malignancy or cancer. Examples include adenomatous growths in ovarian, prostate, lung, or breast tissues, or conditions, for example, dysplastic nevus syndrome, a precursor to malignant melanoma of the skin. Examples also include, abnormal neoplastic, in addition to dysplastic nevus syndromes, polyposis syndromes, prostatic dysplasia, and other such neoplasms, whether the precancerous lesions are clinically identifiable or not.

A “differentially expressed gene transcript”, as used herein, refers to a gene, including an oncogene, transcript that is found in different numbers of copies in different cell or tissue types of an organism having a tumor or cancer, for example, ovarian cancer, prostate cancer, lung cancer, or breast cancer, etc., compared to the numbers of copies or state of the gene transcript found in the cells of the same tissue in a healthy organism, or in the cells of the same tissue in the same organism. Multiple copies of gene transcripts may be found in an organism having the tumor or cancer, while only one, or significantly fewer copies, of the same gene transcript are found in a healthy organism or healthy cells of the same tissue in the same organism, or vice-versa.

A “differentially expressed gene,” can be a target, fingerprint, or pathway gene. For example, a “fingerprint gene”, as used herein, refers to a differentially expressed gene whose expression pattern can be used as a prognostic or diagnostic marker for the evaluation of tumors and cancers, or which can be used to identify compounds useful for the treatment of tumors and cancers, for example, ovarian cancer, prostate cancer, lung cancer, or breast cancer, etc. For example, the effect of a compound on the fingerprint gene expression pattern normally displayed in connection with tumors and cancers can be used to evaluate the efficacy of the compound as a tumor and cancer treatment, or can be used to monitor patients undergoing clinical evaluation for the treatment of tumors and cancer.

A “fingerprint pattern”, as used herein, refers to a pattern generated when the expression pattern of a series (which can range from two up to all the fingerprint genes that exist for a given state) of fingerprint genes is determined. A fingerprint pattern may also be referred to as an “expression profile”. A fingerprint pattern or expression profile can be used in the same diagnostic, prognostic, and compound identification methods as the expression of a single fingerprint gene.

A “target gene”, as used herein, refers to a differentially expressed gene in which modulation of the level of gene expression or of gene product activity prevents and/or ameliorates tumor and cancer, for example, ovarian cancer, symptoms. Thus, compounds that modulate the expression of a target gene, the target genes, or the activity of a target gene product can be used in the diagnosis, treatment or prevention of tumors and cancers. A particular target gene of the present invention is the hepsin gene.

In general, a “gene” is a region on the genome that is capable of being transcribed to an RNA that either has a regulatory function, a catalytic function, and/or encodes a protein. A gene typically has introns and exons, which may organize to produce different RNA splice variants that encode alternative versions of a mature protein. The skilled artisan will appreciate that the present invention encompasses all hepsin-encoding transcripts that may be found, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. A “fragment” of a gene, including an oncogene, can be any portion from the gene, which may or may not represent a functional domain, for example, a catalytic domain, a DNA binding domain, etc. A fragment may preferably include nucleotide sequences that encode for at least 25 contiguous amino acids, and preferably at least about 30, 40, 50, 60, 65, 70, 75 or more contiguous amino acids or any integer thereabout or therebetween.

“Pathway genes”, as used herein, are genes that encode proteins or polypeptides that interact with other gene products involved in tumors and cancers. Pathway genes also can exhibit target gene and/or fingerprint gene characteristics.

A “detectable” RNA expression level, as used herein, means a level that is detectable by standard techniques currently known in the art or those that become standard at some future time, and include for example, differential display, RT (reverse transcriptase)-coupled polymerase chain reaction (PCR), Northern Blot, and/or RNase protection analyses. The degree of differences in expression levels need only be large enough to be visualized or measured via standard characterization techniques, for example, any of the above.

The nucleic acid molecules of the invention, for example, the hepsin gene or its subsequences, can be inserted into a vector, as described below, which will facilitate expression of the insert. The nucleic acid molecules and the polypeptides they encode can be used directly as diagnostic or therapeutic agents, or can be used (directly in the case of the polypeptide or indirectly in the case of a nucleic acid molecule) to generate antibodies that, in turn, are clinically useful as a therapeutic or diagnostic agent. Accordingly, vectors containing the nucleic acid of the invention, cells transfected with these vectors, the polypeptides expressed, and antibodies generated against either the entire polypeptide or an antigenic fragment thereof, are among the aspects of the invention.

As used herein, the term “transformed cell” means a cell into which (or into an ancestor of which) a nucleic acid molecule encoding a polypeptide of the invention has been introduced, by means of, for example, recombinant DNA techniques or viruses.

A “structural gene” is a DNA sequence that is transcribed into messenger RNA (mRNA) which is then translated into a sequence of amino acids characteristic of a specific polypeptide.

An “isolated DNA molecule” is a fragment of DNA that has been separated from the chromosomal or genomic DNA of an organism. Isolation also is defined to connote a degree of separation from original source or surroundings. For example, a cloned DNA molecule encoding an avidin gene is an isolated DNA molecule. Another example of an isolated DNA molecule is a chemically-synthesized DNA molecule, or enzymatically-produced cDNA, that is not integrated in the genomic DNA of an organism. Isolated DNA molecules can be subjected to procedures known in the art to remove contaminants such that the DNA molecule is considered purified, that is towards a more homogeneous state.

“Complementary DNA” (cDNA) is a single-stranded DNA molecule that is formed from an mRNA template by the enzyme reverse transcriptase. Typically, a primer complementary to portions of the mRNA is employed for the initiation of reverse transcription. Those skilled in the art also use the term “cDNA” to refer to a double-stranded DNA molecule that comprises such a single-stranded DNA molecule and its complementary DNA strand.

The term “expression” refers to the biosynthesis of a gene product. For example, in the case of a structural gene, expression involves transcription of the structural gene into mRNA and the translation of mRNA into one or more polypeptides.

The term “amplification” refers to amplification, duplication, multiplication, or multiple expression of nucleic acids or a gene, in vivo or in vitro, yielding about 2.5 fold or more copies. For example, amplification of the hepsin gene resulting in a copy number greater than or equal to 2.5 is deemed to have been amplified. However, an increase in hepsin gene copy number less than 2.5 fold can still be considered as an amplification of the gene.

The term “amplicon” refers to an amplification product containing one or more genes, which can be isolated from a precancerous or a cancerous cell or a tissue. hepsin amplicon is a result of amplification, duplication, multiplication, or multiple expression of nucleic acids or a gene, in vivo or in vitro. “Amplicon”, as defined herein, also include a completely or partially amplified hepsin gene. For example, an amplicon comprising a polynucleotide having at least about 90% sequence identity to SEQ ID NO: 1 or any fragment thereof.

A “cloning vector” is a nucleic acid molecule, for example, a plasmid, cosmid, or bacteriophage that has the capability of replicating autonomously in a host cell. Cloning vectors typically contain (i) one or a small number of restriction endonuclease recognition sites at which foreign DNA sequences can be inserted in a determinable fashion without loss of an essential biological function of the vector, and (ii) a marker gene that is suitable for use in the identification and selection of cells transformed with the cloning vector. Marker genes include genes that provide tetracycline resistance or ampicillin resistance, for example.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers. Such a gene is said to be “operably linked to” or “operatively linked to” the regulatory elements, which means that the regulatory elements control the expression of the gene.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

In eukaryotes, RNA polymerase II catalyzes the transcription of a structural gene to produce mRNA. A DNA molecule can be designed to contain an RNA polymerase II template in which the RNA transcript has a sequence that is complementary to that of a preferred mRNA. The RNA transcript is termed an “antisense RNA”. Antisense RNA molecules inhibit mRNA expression. With respect to a first nucleic acid molecule, a second DNA molecule having a sequence that is complementary to the sequence of the first molecule or the portions thereof is referred to as the “antisense DNA” of the first molecule.

The term “operably linked” is used to describe the connection between regulatory elements and a gene or its coding region. That is, gene expression is typically placed under the control of certain regulatory elements, including constitutive or inducible promoters, tissue-specific regulatory elements, and enhancers. Such a gene is said to be “operably linked to” or “operatively linked to” the regulatory elements.

“Sequence homology” is used to describe the sequence relationships between two or more nucleic acids, polynucleotides, proteins, or polypeptides, and is understood in the context of and in conjunction with the terms including: (a) reference sequence, (b) comparison window, (c) sequence identity, (d) percentage of sequence identity, and (e) substantial identity or “homologous.”

(a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and most preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and most preferably about 100 nucleotides or about 300 nucleotides.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Generally, the comparison window is at least 20 contiguous nucleotides in length, and optionally can be 30, 40, 50, 100, or longer.

Those of skill in the art understand that to avoid a misleadingly high similarity to a reference sequence due to inclusion of gaps in the polynucleotide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 8: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene 73: 237-244 (1988); Higgins and Sharp, CABIOS : 11-13 (1989); Corpet, et al., Nucleic Acids Research 16: 881-90 (1988); Huang, et al., Computer Applications in the Biosciences 8: 1-6 (1992), and Pearson, et al., Methods in Molecular Biology 24: 7-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995). New versions of the above programs or new programs altogether will undoubtedly become available in the future, and can be used with the present invention.

Unless otherwise stated, sequence identity/similarity values provided herein refer to the value obtained using the BLAST 2.0 suite of programs using default parameters. Altschul et al., Nucleic Acids Res. 2:3389-3402 (1997). It is to be understood that default settings of these parameters can be readily changed as needed in the future.

As those ordinary skilled in the art will understand, BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-1 (1993)) low-complexity filters can be employed alone or in combination.

(c) “Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have sequence similarity or similarity. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17 (1988) for example, as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (i) The term “substantial identity” or “homologous” in their various grammatical forms means that a polynucleotide comprises a sequence that has a desired identity, for example, at least 60% identity, preferably at least 70% sequence identity, more preferably at least 80%, still more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, for example,, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

(e) (ii) The terms “substantial identity” or “homologous” in their various grammatical forms in the context of a peptide indicates that a peptide comprises a sequence that has a desired identity, for example, at least 60% identity, preferably at least 70% sequence identity to a reference sequence, more preferably 80%, still more preferably 85%, most preferably at least 90% or 95% sequence identity to the reference sequence over a specified comparison window. Preferably, optimal alignment is conducted using the homology alignment algorithm of Needleman and Wunsch, J. Mol. Biol. 48: 443 (1970). An indication that two peptide sequences are substantially identical is that one peptide is immunologically reactive with antibodies raised against the second peptide, although such cross-reactivity is not required for two polypeptides to be deemed substantially identical. Thus, a peptide is substantially identical to a second peptide, for example, where the two peptides differ only by a conservative substitution. Peptides which are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine.

The term “hepsin” refers to hepsin nucleic acid (DNA and RNA), protein (or polypeptide), and can include their polymorphic variants, alleles, mutants, and interspecies homologs that have (i) substantial nucleotide sequence homology with the nucleotide sequence of the GenBank entry M18930 (human hepsin mRNA, complete cds); or (ii) at least 65% sequence homology with the amino acid sequence of the SWISS-PROT record P05981 (serine protease hepsin); or (iii) substantial nucleotide sequence homology with the nucleotide sequence as set forth in SEQ ID NO: 1; or (iv) substantial sequence homology with the encoded amino acid sequence.

Hepsin polynucleotide or polypeptide sequences are typically from a mammal including, but not limited to, human, rat, mouse, hamster, cow, pig, horse, sheep, or any mammal. A “hepsin polynucleotide” and a “hepsin polypeptide,” may be either naturally occurring, recombinant, or synthetic (for example, via chemical synthesis).

The “level of hepsin mRNA” in a biological sample refers to the amount of mRNA transcribed from a hepsin gene that is present in a cell or a biological sample. The mRNA generally encodes a hepsin protein, often fully functional, although mutations or deletions may be present that alter or eliminate the function of the encoded protein. A “level of hepsin mRNA” need not be quantified, but can simply be detected, for example, via a subjective, visual detection by a human, with or without comparison to a level from a control sample or a level expected of a control sample.

The “level of hepsin protein or polypeptide” in a biological sample refers to the amount of polypeptide translated from a hepsin mRNA that is present in a cell or biological sample. The polypeptide may or may not have hepsin protein activity. A “level of hepsin protein” need not be quantified, but can simply be detected, for example, via a subjective, visual detection by a human, with or without comparison to a level from a control sample or a level expected of a control sample.

A “full length” hepsin protein or nucleic acid refers to a hepsin polypeptide or polynucleotide sequence, or a variant thereof, that contains all of the elements normally contained in one or more naturally occurring, wild type hepsin polynucleotide or polypeptide sequences.

“Biological subject” as used herein refers to a target biological object obtained, reached, or collected in vivo or in situ, including a biological sample, for example, a cell, a tissue, an organ, or body fluid, that contains or is suspected of containing nucleic acids or polypeptides of hepsin. Such biological subjects include, but are not limited to, tissue originated in humans, mice, and rats. Biological subjects may also include sections of the biological subject including tissues, for example, frozen sections taken for histologic purposes. A biological subject is typically of eukaryotic nature, for example, insects, protozoa, birds, fish, reptiles, and preferably a mammal, for example, rat, mouse, cow, dog, guinea pig, or rabbit, and most preferably a primate, for example, chimpanzees or humans.

“Biological sample” as used herein is a biological subject in vivo or in situ, including sample of biological tissue or fluid origin that contains or is suspected of containing nucleic acids or polypeptides of hepsin. Such samples include, but are not limited to, tissue isolated from humans, mice, and rats. Biological samples may also include sections of the biological sample including tissues, for example, frozen sections taken for histologic purposes. A biological sample is typically of an eukaryotic origin, for example, insects, protozoa, birds, fish, reptiles, and preferably a mammal, for example, rat, mouse, cow, dog, guinea pig, or rabbit, and most preferably a primate, for example, chimpanzees or humans.

“Providing a biological subject” means to obtain a biological subject in vivo or in situ, including tissue or cell sample for use in the methods described in the present invention. Most often, this will be done by removing a sample of cells from an animal, but can also be accomplished in vivo or in situ or by using previously isolated cells (for example, isolated by another person, at another time, and/or for another purpose), or by performing the methods of this invention in vivo.

A “control sample” refers to a sample of biological material representative of healthy, cancer-free animals. The level of hepsin or hepsin gene copy number in a control sample is desirably typical of the general population of normal, cancer-free animals of the same species. This sample either can be collected from an animal for the purpose of being used in the methods described in the present invention or, it can be any biological material representative of normal, cancer-free animals obtained for other reasons but nonetheless suitable for use in the methods of this invention. A control sample can also be obtained from normal tissue from the animal that has cancer or is suspected of having cancer. A control sample also can refer to a given level of hepsin representative of the cancer-free population, that has been previously established based on measurements from normal, cancer-free animals. Alternatively, a biological control sample can refer to a sample that is obtained from a different individual or be a normalized value based on baseline values found in a population. Further, a control sample can be defined by a specific age, sex, ethnicity or other demographic parameters. In some situations, the control is implicit in the particular measurement. For example, a detection method that can only detect hepsin or hepsin gene copy number when a level higher than that typical of a normal, cancer-free animal is present, for example, an immunohistochemical assay, is considered to be assessing the hepsin level in or hepsin gene copy number comparison to the control level or hepsin gene copy number, as the control level or the copy number is natural and known in the assay.

“Data” refers to information obtained that relates to “Biological Sample” or “Control Sample”, as described above, wherein the information is applied in generating a test level for diagnostics, prevention, monitoring or therapeutic use. The present invention relates to methods for comparing and compiling data wherein the data is stored in electronic or paper formats. Electronic format can be selected from the group consisting of electronic mail, disk, compact disk (CD), digital versatile disk (DVD), memory card, memory chip, ROM or RAM, magnetic optical disk, tape, video, video clip, microfilm, internet, shared network, shared server and the like; wherein data is displayed, transmitted or analyzed via electronic transmission, video display, telecommunication, or by using any of the above stored formats; wherein data is compared and compiled at the site of sampling specimens or at a location where the data is transported following a process as described above.

“Overexpression” of a hepsin gene or an “increased,” or “elevated,” level of a hepsin polynucleotide or protein refers to a level of hepsin polynucleotide or polypeptide that, in comparison with a control level of hepsin, is detectably higher. Comparison may be carried out by statistical analyses on numeric measurements of the expression; or, it may be done through visual examination of experimental results by qualified researchers.

A level of hepsin polypeptide or polynucleotide that is “expected” in a control sample refers to a level that represents a typical, cancer-free sample, and from which an elevated, or diagnostic, presence of hepsin polypeptide or polynucleotide can be distinguished. Preferably, an “expected” level will be controlled for such factors as the age, sex, medical history, etc. of the mammal, as well as for the particular biological subject being tested.

The phrase “functional effects” in the context of an assay or assays for testing compounds that modulate hepsin activity includes the determination of any parameter that is indirectly or directly under the influence of hepsin, for example, a functional, physical, or chemical effect, for example, the protease activity, the ability to induce gene amplification or overexpression in cancer cells, and to aggravate cancer cell proliferation. “Functional effects” include in vitro, in vivo, and ex vivo activities.

“Determining the functional effect” refers to assaying for a compound that increases or decreases a parameter that is indirectly or directly under the influence of hepsin, for example, functional, physical, and chemical effects. Such functional effects can be measured by any means known to those skilled in the art, for example, changes in spectroscopic characteristics (for example, fluorescence, absorbance, refractive index), hydrodynamic (for example, shape), chromatographic, or solubility properties for the protein, measuring inducible markers or transcriptional activation of hepsin; measuring binding activity or binding assays, for example, substrate binding, and measuring cellular proliferation; measuring signal transduction; or measuring cellular transformation.

“Inhibitors,” “activators,” “modulators,” and “regulators” refer to molecules that activate, inhibit, modulate and/or regulate an identified function. For example, referring to hepsin activity, such molecules may be identified using in vitro and in vivo assays of hepsin. Inhibitors are compounds that partially or totally block hepsin activity, decrease, prevent, or delay its activation, or desensitize its cellular response. This may be accomplished by binding to hepsin proteins directly or via other intermediate molecules. An antagonist of hepsin is considered to be such an inhibitor. Activators are compounds that bind to hepsin protein directly or via other intermediate molecules, thereby increasing or enhancing its activity, stimulating or accelerating its activation, or sensitizing its cellular response. An agonist of hepsin is considered to be such an activator. A modulator can be an inhibitor or activator. A modulator may or may not bind hepsin or its protein directly; it affects or changes the activity or activation of hepsin or the cellular sensitivity to hepsin. A modulator also may be a compound, for example, a small molecule, that inhibits transcription of hepsin mRNA.

The group of inhibitors, activators and modulators of this invention also includes genetically modified versions of hepsin, for example, versions with altered activity. The group thus is inclusive of the naturally occurring protein as well as synthetic ligands, antagonists, agonists, antibodies, small chemical molecules and the like.

“Assays for inhibitors, activators, or modulators” refer to experimental procedures including, for example, expressing hepsin in vitro, in cells, applying putative inhibitor, activator, or modulator compounds, and then determining the functional effects on hepsin activity, as described above. Samples that contain or are suspected of containing hepsin are treated with a potential activator, inhibitor, or modulator. The extent of activation, inhibition, or change is examined by comparing the activity measurement from the samples of interest to control samples. A threshold level is established to assess activation or inhibition. For example, inhibition of a hepsin polypeptide is considered achieved when the hepsin activity value relative to the control is 80% or lower. Similarly, activation of a hepsin polypeptide is considered achieved when the hepsin activity value relative to the control is two or more fold higher.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. Various levels of purity may be applied as needed according to this invention in the different methodologies set forth herein; the customary purity standards known in the art may be used if no standard is otherwise specified.

An “isolated nucleic acid molecule” can refer to a nucleic acid molecule, depending upon the circumstance, that is separated from the 5′ and 3′ coding sequences of genes or gene fragments contiguous in the naturally occurring genome of an organism. The term “isolated nucleic acid molecule” also includes nucleic acid molecules which are not naturally occurring, for example, nucleic acid molecules created by recombinant DNA techniques.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., Nucleic Acid Res. 19:081 (1991); Ohtsuka et al., J. Biol. Chem. 260:2600-2608 (1985); Rossolini et al., Mol. Cell. Probes 8:91-98 (1994)). The term nucleic acid is used interchangeably with gene, cDNA, mRNA, oligonucleotide, and polynucleotide.

A “host cell” is a naturally occurring cell or a transformed cell that contains an expression vector and supports the replication or expression of the expression vector. Host cells may be cultured cells, explants, cells in vivo, and the like. Host cells may be prokaryotic cells, for example, E. coli, or eukaryotic cells, for example, yeast, insect, amphibian, or mammalian cells, for example, CHO, HeLa, and the like.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, γ-carboxyglutamate, and O-phosphoserine, phosphotheorine. “Amino acid analogs” refer to compounds that have the same basic chemical structure as a naturally occurring amino acid, i.e., a carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid. Amino acids and analogs are well known in the art.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” apply to both amino acid and nucleic acid sequences. With respect to particular nucleic acid sequences, conservatively modified variants refers to those nucleic acids which encode identical or similar amino acid sequences and include degenerate sequences. For example, the codons GCA, GCC, GCG and GCU all encode alanine. Thus, at every amino acid position where an alanine is specified, any of these codons can be used interchangeably in constructing a corresponding nucleotide sequence. The resulting nucleic acid variants are conservatively modified variants, since they encode the same protein (assuming that is the only alternation in the sequence). One skilled in the art recognizes that each codon in a nucleic acid, except for AUG (sole codon for methionine) and TGG (tryptophan), can be modified conservatively to yield a functionally-identical peptide or protein molecule.

As to amino acid sequences, one skilled in the art will recognize that substitutions, deletions, or additions to a polypeptide or protein sequence which alter, add or delete a single amino acid or a small number (typically less than ten) of amino acids is a “conservatively modified variant” where the alteration results in the substitution of an amino acid with a chemically similar amino acid. Conservative substitutions are well known in the art and include, for example, the changes of: alanine to serine; arginine to lysine; asparigine to glutamine or histidine; aspartate to glutamate; cysteine to serine; glutamine to asparigine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine.

The terms “protein”, “peptide” and “polypeptide” are used herein to describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The polypeptides of the invention can be chemically synthesized or synthesized by recombinant DNA methods; or, they can be purified from tissues in which they are naturally expressed, according to standard biochemical methods of purification.

Also included in the invention are “functional polypeptides,” which possess one or more of the biological functions or activities of a protein or polypeptide of the invention. These functions or activities include the ability to bind some or all of the proteins which normally bind to hepsin protein.

The functional polypeptides may contain a primary amino acid sequence that has been modified from that considered to be the standard sequence of hepsin described herein. Preferably these modifications are conservative amino acid substitutions, as described herein.

A “label” or a “detectable moiety” is a composition that when linked with the nucleic acid or protein molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. A “labeled nucleic acid or oliionucleotide Probe” is one that is bound, either covalently, through a linker or a chemical bond, or noncovalently, through ionic, van der Waals, electrostatic, hydrophobic interactions, or hydrogen bonds, to a label such that the presence of the nucleic acid or probe may be detected by detecting the presence of the label bound to the nucleic acid or probe.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

The phrase “stringent hybridization conditions” refers to conditions under which a probe will hybridize to its target complementary sequence, typically in a complex mixture of nucleic acids, but to no other sequences. Stringent conditions are sequence-dependent and circumstance-dependent; for example, longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). In the context of the present invention, as used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other.

Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at TR, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

Exemplary, non-limiting stringent hybridization conditions can be as following: 50% formamide, 5×SSC, and 1% SDS, incubating at 42° C., or, 5×SSC, 1 SDS, incubating at 65° C., with wash in 0.2×SSC, and 0.1% SDS at 65° C. Alternative conditions include, for example, conditions at least as stringent as hybridization at 68° C. for 20 hours, followed by washing in 2×SSC, 0.1% SDS, twice for 30 minutes at 55° C. and three times for 15 minutes at 60° C. Another alternative set of conditions is hybridization in 6×SSC at about 45° C., followed by one or more washes in 0.2×SSC, 0.1% SDS at 50-65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec.-2 min., an annealing phase lasting 30 sec.-2 min., and an extension phase of about 72° C. for 1 -2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 2 kD) and one “heavy” chain (about 0-70 kD).

Antibodies exist, for example, as intact immunoglobulins or as a number of well-characterized fragments produced by digestion with various peptidases. While various antibody fragments are defined in terms of the digestion of an intact antibody, one of skilled in the art will appreciate that such fragments may be synthesized de novo chemically or via recombinant DNA methodologies. Thus, the term antibody, as used herein, also includes antibody fragments produced by the modification of whole antibodies, those synthesized de novo using recombinant DNA methodologies (for example, single chain Fv), humanized antibodies, and those identified using phage display libraries (see, for example, Knappik et al. J Mol Biol. 2000 296:57-86; McCafferty et al., Nature 348:2-4 (1990)), for example. For preparation of antibodies—recombinant, monoclonal, or polyclonal antibodies—any technique known in the art can be used in this invention (see, for example, Kohler & Milstein, Nature 26:49-497 (1997); Kozbor et al., Immunology Today 4: 72 (1983); Cole et al., pp. 77-96 in Monoclonal Antibodies and Cancer Therapy, Alan R. Liss, Inc. (1998)).

Techniques for the production of single chain antibodies (See U.S. Pat. No. 4,946,778) can be adapted to produce antibodies to polypeptides of this invention. Transgenic mice, or other organisms, for example, other mammals, may be used to express humanized antibodies. Phage display technology can also be used to identify antibodies and heteromeric Fab fragments that specifically bind to selected antigens (see, for example, McCafferty et al., Nature 348:2-4 (1990); Marks et al., Biotechnology: 779-783 (1992)).

An “anti-hepsin” antibody is an antibody or antibody fragment that specifically binds a polypeptide encoded by a hepsin gene, cDNA, or a subsequence thereof.

The term “immunoassay” is an assay that utilizes the binding interaction between an antibody and an antigen. Typically, an immunoassay uses the specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at a level at least two times the background and do not substantially bind in a significant amount to other proteins present in the sample. Specific binding to an antibody under such conditions may require an antibody that is selected for its specificity for a particular protein. For example, antibodies raised to a particular hepsin polypeptide can be selected to obtain only those antibodies that are specifically immunoreactive with the hepsin polypeptide, respectively, and not with other proteins, except for polymorphic variants, orthologs, and alleles of the specific hepsin polypeptide. In addition, antibodies raised to a particular hepsin polypeptide ortholog can be selected to obtain only those antibodies that are specifically immunoreactive with the hepsin polypeptide ortholog, respectively, and not with other orthologous proteins, except for polymorphic variants, mutants, and alleles of the hepsin polypeptide ortholog. This selection may be achieved by subtracting out antibodies that cross-react with desired hepsin molecule, as appropriate. A variety of immunoassay formats may be used to select antibodies specifically immunoreactive with a particular protein. For example, solid-phase ELISA immunoassays are routinely used to select antibodies specifically immunoreactive with a protein. See, for example, Harlow & Lane, Antibodies, A Laboratory Manual (1988), for a description of immunoassay formats and conditions that can be used to determine specific immunoreactivity.

The phrase “selectively associates with” refers to the ability of a nucleic acid to “selectively hybridize” with another as defined supra, or the ability of an antibody to “selectively (or specifically) bind” to a protein, as defined supra.

“siRNA” refers to small interfering RNAs, that are capable of causing interference and can cause post-transcriptional silencing of specific genes in cells, for example, mammalian cells (including human cells) and in the body, for example, mammalian bodies (including humans). The phenomenon of RNA interference is described and discussed in Bass, Nature 411: 428-29 (2001); Elbahir et al., Nature 411: 494-98 (2001); and Fire et al., Nature 391: 806-11 (1998), where methods of making interfering RNA also are discussed. The siRNAs based upon the sequence disclosed herein (for example, GenBank Accession No. M18930 for hepsin mRNA sequence) is less than 100 base pairs (“bps”) in length and constituency and preferably is about 30 bps or shorter, and can be made by approaches known in the art, including the use of complementary DNA strands or synthetic approaches. The siRNAs are capable of causing interference and can cause post-transcriptional silencing of specific genes in cells, for example, mammalian cells (including human cells) and in the body, for example, mammalian bodies (including humans). Exemplary siRNAs according to the invention could have up to 29 bps, 25 bps, 22 bps, 21 bps, 20 bps, 15 bps, 10 bps, 5 bps or any integer thereabout or therebetween.

The term “transgene” refers to a nucleic acid sequence encoding, for example, one of the hepsin polypeptides, or an antisense transcript thereto, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (for example, it is inserted at a location which differs from that of the natural gene or its insertion results in a knockout). A transgene can include one or more transcriptional regulatory sequences and any other nucleic acid, (for example, as intron), that may be necessary for optimal expression of a selected nucleic acid.

A “transgenic animal” refers to any animal, preferably a non-human mammal, transgenic and chimeric animals of most vertebrate species. Such species include, but are not limited to, non-human mammals, including rodents, for example, mice and rats, rabbits, bird or an amphibian, ovines, for example, sheep and goats, porcines, for example, pigs, and bovines, for example, cattle and buffalo in which one or more of the cells of the animal contain heterologous nucleic acid introduced by way of human intervention, for example, by transgenic techniques well known in the art. The nucleic acid is introduced into the cell, directly or indirectly by introduction into a precursor of the cell, by way of deliberate genetic manipulation, for example, by microinjection or by infection with a recombinant virus. The term genetic manipulation does not include classical cross-breeding, or sexual fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the hepsin proteins, for example, either agonistic or antagonistic forms. However, transgenic animals in which the recombinant hepsin gene is silent are also contemplated. Moreover, “transgenic animal” also includes those recombinant animals in which gene disruption of one or more hepsin gene is caused by human intervention, including both recombination and antisense techniques.

Methods of obtaining transgenic animals are described in, for example, Puhler, A., Ed., Genetic Engineering of animals, VCH Pub., 1993; Murphy and Carter, Eds., Transgenesis Techniques: Principles and Protocols (Methods in Molecular Biology, Vol. 18), 1993; and Pinkert, C A, Ed., Transgenic Animal Technology: A Laboratory Handbook, Academic Press, 1994.

The term “knockout construct” refers to a nucleotide sequence that is designed to decrease or suppress expression of a polypeptide encoded by an endogenous gene in one or more cells of a mammal. The nucleotide sequence used as the knockout construct is typically comprised of (1) DNA from some portion of the endogenous gene (one or more exon sequences, intron sequences, and/or promoter sequences) to be suppressed and (2) a marker sequence used to detect the presence of the knockout construct in the cell. The knockout construct can be inserted into a cell containing the endogenous gene to be knocked out. The knockout construct can then integrate with one or both alleles of an endogenous gene, for example, hepsin gene, and such integration of the knockout construct can prevent or interrupt transcription of the full-length endogenous gene. Integration of the knockout construct into the cellular chromosomal DNA is typically accomplished via homologous recombination (i.e., regions of the knockout construct that are homologous or complementary to endogenous DNA sequences can hybridize to each other when the knockout construct is inserted into the cell; these regions can then recombine so that the knockout construct is incorporated into the corresponding position of the endogenous DNA).

By “transgenic” is meant any mammal that includes a nucleic acid sequence, which is inserted into a cell and becomes a part of the genome of the animal that develops from that cell. Such a transgene may be partly or entirely heterologous to the transgenic animal.

Thus, for example, substitution of the naturally occurring hepsin gene for a gene from a second species results in an animal that produces the protein of the second species. Substitution of the naturally occurring gene for a gene having a mutation results in an animal that produces the mutated protein. A transgenic mouse expressing the human hepsin protein can be generated by direct replacement of the mouse hepsin subunit with the human gene. These transgenic animals can be critical for drug antagonist studies on animal models for human diseases, and for eventual treatment of disorders or diseases associated with the respective genes. Transgenic mice carrying these mutations will be extremely useful in studying this disease.

A transgenic animal carrying a “knockout” of hepsin gene, would be useful for the establishment of a non-human model for diseases involving such proteins, and to distinguish between the activities of the different hepsin proteins in an in vivo system. “Knockout mice” refers to mice whose native or endogenous hepsin allele or alleles have been disrupted by homologous recombination and which produce no functional hepsin of their own. Knockout mice may be produced in accordance with techniques known in the art, for example, Thomas, et al., (1999) Immunol. 163:978-84; Kanakaraj, et al. (1998) J. Exp. Med. 187:2073-9; or Yeh, et al., (1997) Immunity 7:715-725.

Hepsin: A trypsin-like serine protease: The GenBank entry M18930 Homo sapiens, hepsin gene is as shown below:

| | |
|---|---|
| 1 | TCGAGCCCGC TTTCCAGGGA CCCTACCTGA GGGCCCACAG GTGAGGCAGC CTGGCCTAGC |
| 61 | AGGCCCCACG CCACCGCCTC TGCCTCCAGG CCGCCCGCTG CTGCGGGGCC ACCATGCTCC |
| 121 | TGCCCAGGCC TGGAGACTGA CCCGACCCCG GCACTACCTC GAGGCTCCGC CCCCACCTGC |
| 181 | TGGACCCCAG GGTCCCACCC TGGCCCAGGA GGTCAGCCAG GGAATCATTA ACAAGAGGCA |
| 241 | GTGACATGGC GCAGAAGGAG GGTGGCCGGA CTGTGCCATG CTGCTCCAGA CCCAAGGTGG |
| 301 | CAGCTCTCAC TGCGGGGACC CTGCTACTTC TGACAGCCAT CGGGGCGGCA TCCTGGGCCA |
| 361 | TTGTGGCTGT TCTCCTCAGG AGTGACCAGG AGCCGCTGTA CCCAGTGCAG GTCAGCTCTG |
| 421 | CGGACGCTCG GCTCATGGTC TTTGACAAGA CGGAAGGGAC GTGGCGGCTG CTGTGCTCCT |
| 481 | CGCGCTCCAA CGCCAGGGTA GCCGGACTCA GCTGCGAGGA GATGGGCTTC CTCAGGGCAC |
| 541 | TGACCCACTC CGAGCTGGAC GTGCGAACGG CGGGCGCCAA TGGCACGTCG GGCTTCTTCT |
| 601 | GTGTGGACGA GGGGAGGCTG CCCCACACCC AGAGGCTGCT GGAGGTCATC TCCGTGTGTG |
| 661 | ATTGCCCCAG AGGCCGTTTC TTGGCCGCCA TCTGCCAAGA CTGTGGCCGC AGGAAGCTGC |
| 721 | CCGTGGACCG CATCGTGGGA GGCCGGGACA CCAGCTTGGG CCGGTGGCCG TGGCAAGTCA |
| 781 | GCCTTCGCTA TGATGGAGCA CACCTCTGTG GGGGATCCCT GCTCTCCGGG GACTGGGTGC |
| 841 | TGACAGCCGC CCACTGCTTC CCGGAGCGGA ACCGGGTCCT GTCCCGATGG CGAGTGTTTG |
| 901 | CCGGTGCCGT GGCCCAGGCC TCTCCCCACG GTCTGCAGCT GGGGGTGCAG GCTGTGGTCT |
| 961 | ACCACGGGGG CTATCTTCCC TTTCGGGACC CCAACAGCGA GGAGAACAGC AACGATATTG |
| 1021 | CCCTGGTCCA CCTCTCCAGT CCCCTGCCCC TCACAGAATA CATCCAGCCT GTGTGCCTCC |
| 1081 | CAGCTGCCGG CCAGGCCCTG GTGGATGGCA AGATCTGTAC CGTGACGGGC TGGGGCAACA |
| 1141 | CGCAGTACTA TGGCCAACAG GCCGGGGTAC TCCAGGAGGC TCGAGTCCCC ATAATCAGCA |
| 1201 | ATGATGTCTG CAATGGCGCT GACTTCTATG GAAACCAGAT CAAGCCCAAG ATGTTCTGTG |
| 1261 | CTGGCTACCC CGAGGGTGGC ATTGATGCCT GCCAGGGCGA CAGCGGTGGT CCCTTTGTGT |
| 1321 | GTGAGGACAG CATCTCTCGG ACGCCACGTT GGCGGCTGTG TGGCATTGTG AGTTGGGGCA |
| 1381 | CTGGCTGTGC CCTGGCCCAG AAGCCAGGCG TCTACACCAA AGTCAGTGAC TTCCGGGAGT |
| 1441 | GGATCTTCCA GGCCATAAAG ACTCACTCCG AAGCCAGCGG CATGGTGACC CAGCTCTGAC |
| 1501 | CGGTGGCTTC TCGCTGCGCA GCCTCCAGGG CCCGAGGTGA TCCCGGTGGT GGGATCCACG |
| 1561 | CTGGGCCGAG GATGGGACGT TTTTCTTCTT GGGCCCGGTC CACAGGTCCA AGGACACCCT |
| 1621 | CCCTCCAGGG TCCTCTCTTC CACAGTGGCG GGCCCACTCA GCCCCGAGAC CACCCAACCT |
| 1681 | CACCCTCCTG ACCCCCATGT AAATATTGTT CTGCTGTCTG GGACTCCTGT CTAGGTGCCC |
| 1741 | CTGATGATGG GATGCTCTTT AAATAATAAA GATGGTTTTG ATT |

Hepsin Protein Sequence:

/protein_id=“AAA36013.1”

“MAQKEGGRTVPCCSRPKVAALTAGTLLLLTAIGAASWAIVAVLLRSDQE
PLYPVQVSSADARLMVFDKTEGTWRLLCSSRSNARVAGLSCEEMGFLRAL
THSELDVRTAGANGTSGFFCVDEGRLPHTQRLLEVISVCDCPRGRFLAAI
CQDCGRRKLPVDRIVGGRDTSLGRWPWQVSLRYDGAHLCGGSLLSGDWVL
TAAHCFPERNRVLSRWRVFAGAVAQASPHGLQLGVQAVVYHGGYLPFRDP
NSEENSNDIALVHLSSPLPLTEYIQPVCLPAAGQALVDGKICTVTGWGNT
QYYGQQAGVLQEARVPIISNDVCNGADFYGNQIKPKMFCAGYPEGGIDAC
QGDSGGPFVCEDSISRTPRWRLCGIVSWGTGCALAQKPGVYTKVSDFREW
IFQAIKTHSEASGMVTQL”

Human chromosome region 19q13 is one of the most frequently amplified regions in human ovarian cancer. In a process of characterizing one of the 19q13 amplicons, hepsin was found amplified in over 17% (5/29 samples) in ovarian tumor samples (see Table 2) and in over 37% (3/8 samples) in ovarian cell lines (see Table 4). Study shown that this amplification is usually associated with aggressive histologic types. Amplification of tumor-promoting gene(s) located on 19q13 may play an important role in the development and/or progression of a substantial proportion of primary ovarian or prostate cancers, particularly those of the invasive histology.

Hepsin was found by DNA microarray analysis of human ovarian tumor for DNA amplification using the methods described elsewhere. See, for example, U.S. Pat. No 6,232,068; Pollack et al., Nat. Genet. 23(1):41-46, 1999. Further analysis provided evidence that hepsin is at the epicenter of amplification region.

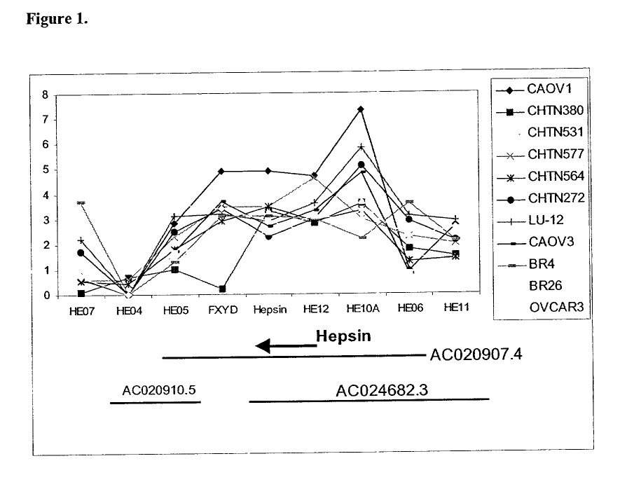

Amplified cell lines or tumors (ovarian and other types) were examined for DNA copy number of nearby genes and DNA sequences that map to the boundaries of the amplified regions. TaqMan epicenter data for hepsin is shown in FIG. 1.

The corresponding genomic DNA sequence from the human genome project was analyzed for the presence of genes. Hepsin was found at the epicenter. Overall hepsin was found amplified in over 17% of human ovarian tumors.

Quantitative RT-PCR analysis with Taqman probes showed that hepsin was found overexpressed in over 80% of human ovarian tumor samples (4/5 and 25/29 samples, see Tables 1 and 2, respectively) and over 70% in prostate tumor samples (10/14 samples, see Table 3). All amplified ovarian tumors overexpress hepsin mRNA (see Table 2).

TABLE 1
Expression of hepsin in ovarian tumor.

| | TUMOR | RELATIVE HEPSIN mRNA |
|---|---|---|
| IDENTIFIER | OR NORMAL | LEVEL |
| CHTN 544 | ovarian tumor | 0.31 |
| CHTN 545 (NAT to 544) | NAT, ovary | 1 |
| CHTN 579 | ovarian tumor | 11 |
| CHTN 578 (NAT to 579) | NAT, ovary | 1 |
| CHTN 749 | ovarian tumor | 10 |
| CHTN 750 (NAT to 749) | NAT, ovary | 1 |
| CHTN 478 | ovarian tumor | 138 |
| CHTN 479 (NAT to 478) | NAT, ovary | 1 |
| CHTN 740 | ovarian tumor | 41 |
| CHTN 741 (NAT to 740) | NAT, ovary | 1 |

TABLE 2
Amplification and overexpression frequency of hepsin in ovarian tumor
samples and ovarian tumor cell lines.

| | | HEPSIN DNA | RELATIVE |
|---|---|---|---|
| | TUMOR | COPY | HEPSIN MRNA |
| IDENTIFIER | OR NORMAL | NUMBER | LEVEL |
| CHTN 272 | ovarian tumor | 2.7 | 7.6 |
| CHTN 273 | ovarian tumor | 0.51 | 121 |
| CHTN 276 | ovarian tumor | 1.8 | 0.33 |
| CHTN 277 | ovarian tumor | 0.61 | 156 |
| CHTN 279 | ovarian tumor | 0.61 | 64 |
| CHTN 281 | ovarian tumor | 0.19 | 578 |
| CHTN 282 | ovarian tumor | 0.32 | 29 |
| CHTN 284 | ovarian tumor | 0.66 | 0.61 |
| CHTN 558 | ovarian tumor | 1.7 | 515 |
| CHTN 652 | ovarian tumor | 2.1 | 29 |
| CHTN 577 | ovarian tumor | 3.5 | 399 |
| CHTN 564 | ovarian tumor | 3.5 | 523 |
| CHTN 552 | ovarian tumor | 0.67 | 0.19 |
| CHTN 531 | ovarian tumor | 3.3 | 104 |
| CHTN 380 | ovarian tumor | 3.3 | 25 |
| CHTN 653 | ovarian tumor | 0.7 | 320 |
| CHTN 274 | ovarian tumor | 0.55 | 25 |
| CHTN 275 | ovarian tumor | 1.9 | 2.1 |
| CHTN 478 | ovarian tumor | 0.56 | 115 |
| CHTN 100 | ovarian tumor | 0.71 | 367 |
| CHTN 286 | ovarian tumor | 0.39 | 6.6 |
| CHTN 285 | ovarian tumor | 0.78 | 190 |
| CHTN 289 | ovarian tumor | 0.98 | 84 |
| CHTN 290 | ovarian tumor | 0.78 | 357 |
| CHTN 291 | ovarian tumor | 0.46 | 6.9 |
| CHTN 310 | ovarian tumor | 0.72 | 112 |
| CHTN 312 | ovarian tumor | 0.41 | 221 |
| CHTN 313 | ovarian tumor | 1.2 | 342 |
| CHTN 315 | ovarian tumor | 0.38 | 54 |
| Normal human | normal | N.D. | 1 |
| ovary tissue | | | |
| CAOV1 | ovarian tumor | 4.9 | 9.6 |
| | cell line | | |
| CAOV3 | ovarian tumor | 3.3 | 39 |
| | cell line | | |
| CAOV4 | ovarian tumor | 0.82 | 68 |
| | cell line | | |
| OVCAR3 | ovarian tumor | 2.5 | 8 |
| | cell line | | |
| colo316 | ovarian tumor | 0.47 | 0.006 |
| | cell line | | |
| SW626 | ovarian tumor | 2.3 | 6.7 |
| | cell line | | |
| ES2 | ovarian tumor | 0.45 | 0.11 |
| | cell line | | |
| colo704 | ovarian tumor | N.D. | 0.069 |
| | cell line | | |
| SKOV3 | ovarian tumor | 1.8 | 0.1 |
| | cell line | | |

N.D. = Not determined

The folds of amplification and folds of overexpression were measured by Taqman and RT-Taqman respectively using hepsin specific fluorogenic Taqman probes. There is a good correlation between and amplification and overexpression (see Tables 1 and 2).

TABLE 3
Expression of hepsin mRNA in prostate tumor tissues.

| | | RELATIVE HEPSIN |
|---|---|---|
| | TUMOR TISSUE OR | mRNA EXPRESSION |
| IDENTIFIER | NORMAL TISSUE | LEVEL |
| 480 | prostate tumor | 0.26 |
| 484 | prostate tumor | 0.61 |
| 486 | prostate tumor | 19 |
| WA4-1 | prostate tumor, metastatic | 80 |
| WA4-3 | prostate tumor, metastatic | 78 |
| WA5-1 | prostate tumor, metastatic | 68 |
| WA13-1 | prostate tumor, metastatic | 16 |
| WA5-3 | prostate tumor, metastatic | 14 |
| WA5-4 | prostate tumor, metastatic | 7.7 |
| WA20-10 | prostate tumor, metastatic | 23 |
| WA20-45 | prostate tumor, metastatic | 89 |
| PP2 | prostate tumor | 0.41 |
| PP8 | prostate tumor | 17 |
| PP12 | prostate tumor | 0.37 |
| Normal Prostate | normal | 1.0 |
| Tissue | | |

TABLE 4
Amplification of hepsin gene in various tumor types.

| | | HEPSIN | TOTAL # | |
|---|---|---|---|---|
| | | GENE | OF | |
| | AMPLIFIED | COPY | TUMORS | AMP. |
| TUMOR TYPE | SAMPLE | # | SCREENED | FREQUENCY |
| Ovarian tumors | CHTN 272 | 2.7 | 29 | 17% (5/29) |
| | CHTN 380 | 3.3 | | |
| | CHTN 531 | 3.3 | | |
| | CHTN 564 | 3.5 | | |
| | CHTN 577 | 3.5 | | |
| Ovarian tumor | CAOV1 | 4.9 | 8 | 38% (⅜) |
| cell lines | CAOV3 | 2.7 | | |
| | OVCAR3 | 2.5 | | |
| Lung tumors | LU-12 | 2.9 | 33 | 3% (1/33) |
| Breast tumors | BR4 | 3.6 | 35 | 6% (2/35) |
| | BR26 | 2.7 | | |
| Prostate tumors | | | 16 | 0% (0/16) |

More details on the possible role of hespin in tumorigenesis are discussed in the sections below.

Amplification of Hepsin Gene in Tumors:

The presence of a target gene that has undergone amplification in tumors is evaluated by determining the copy number of the target genes, i.e., the number of DNA sequences in a cell encoding the target protein. Generally, a normal cell has two copies of a given autosomal gene. The copy number can be increased, however, by gene amplification or duplication, for example, in cancer cells, or reduced by deletion. Methods of evaluating the copy number of a particular gene are well known in the art, and include, inter alia, hybridization and amplification based assays.

Any of a number of hybridization based assays can be used to detect the copy number of the hepsin gene in the cells of a biological sample. One such method is Southern blot (see Ausubel et al., or Sambrook et al., supra), where the genomic DNA is typically fragmented, separated electrophoretically, transferred to a membrane, and subsequently hybridized to a hepsin specific probe. Comparison of the intensity of the hybridization signal from the probe for the target region with a signal from a control probe from a region of normal nonamplified, single-copied genomic DNA in the same genome provides an estimate of the relative hepsin copy number, corresponding to the specific probe used. An increased signal compared to control represents the presence of amplification.

A methodology for determining the copy number of the hepsin gene in a sample is in situ hybridization, for example, fluorescence in situ hybridization (FISH) (see Angerer, 1987 Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Another alternative methodology for determining number of DNA copies is comparative genomic hybridization (CGH). In comparative genomic hybridization methods, a “test” collection of nucleic acids is labeled with a first label, while a second collection (for example, from a normal cell or tissue) is labeled with a second label. The ratio of hybridization of the nucleic acids is determined by the ratio of the first and second labels binding to each fiber in an array. Differences in the ratio of the signals from the two labels, for example, due to gene amplification in the test collection, is detected and the ratio provides a measure of the hepsin gene copy number, corresponding to the specific probe used. A cytogenetic representation of DNA copy-number variation can be generated by CGH, which provides fluorescence ratios along the length of chromosomes from differentially labeled test and reference genomic DNAs.

Hybridization protocols suitable for use with the methods of the invention are described, for example, in Albertson (1984) EMBO J. 3:1227-1234; Pinkel (1988) Proc. Natl. Acad. Sci. USA 85:9138-9142; EPO Pub. No. 430:402; Methods in Molecular Biology, Vol. 33: In Situ Hybridization Protocols, Choo, ed., Humana Press, Totowa, N.J. (1994).

Amplification-based assays also can be used to measure the copy number of the hepsin gene. In such assays, the corresponding hepsin nucleic acid sequences act as a template in an amplification reaction (for example, Polymerase Chain Reaction or PCR). In a quantitative amplification, the amount of amplification product will be proportional to the amount of template in the original sample. Comparison to appropriate controls provides a measure of the copy number of the hepsin gene, corresponding to the specific probe used, according to the principle discussed above. Methods of real-time quantitative PCR using Taqman probes are well known to in the art. Detailed protocols for real-time quantitative PCR are provided, for example, for RNA in: Gibson et al., 1996, A novel method for real time quantitative RT-PCR. Genome Res. 10:995-1001; and for DNA in: Heid et al., 1996, Real time quantitative PCR. Genome Res. 10:986-994.

A TaqMan-based assay can also be used to quantify hepsin polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, http://www2.perkin-elmer.com).

Other suitable amplification methods include, but are not limited to, ligase chain reaction (LCR) (see, Wu and Wallace, 1989, Genomics 4: 560; Landegren et al., 1988 Science 241: 1077; and Barringer et al., 1990, Gene 89: 117), transcription amplification (Kwoh et al., 1989, Proc. Natl. Acad. Sci. USA 86: 1173), self-sustained sequence replication (Guatelli et al., 1990, Proc. Nat. Acad. Sci. USA 87: 1874), dot PCR, and linker adapter PCR, etc.

One powerful method for determining DNA copy numbers uses microarray-based platforms. Microarray technology may be used because it offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of a microarray method can be found in the literature. See, for example, U.S. Pat. No. 6,232,068; Pollack et al., Nat Genet, 1999, 23(1):41-6.

As demonstrated in the Examples set forth herein, the hepsin gene is frequently amplified in certain cancers, particularly ovarian cancers; and it resides at the epicenter of the amplified chromosome region. All samples showing hepsin gene amplification in Table 2 also demonstrate overexpression of hepsin mRNA. The hepsin gene has these characteristic features of overexpression, amplification, and the correlation between the two, and these features are shared with other well studied oncogenes (Yoshimoto et al., 1986, JPN J Cancer Res, 77(6):540-5; Knuutila et al., Am J Pathol 1998 152(5):1107-23). The hepsin genes are accordingly used in the present invention as a target for cancer diagnosis and treatment.

Frequent Overexpression of Hepsin Gene in Tumors:

The expression levels of the hepsin gene in a variety of tumors were examined. As demonstrated in the examples infra, hepsin gene is overexpressed in ovarian and prostate cancer cell lines. Detection and quantification of the hepsin gene expression may be carried out through direct hybridization based assays or amplification based assays. The hybridization based techniques for measuring gene transcript are known to those skilled in the art (Sambrook et al., 1989. Molecular Cloning: A Laboratory Manual, 2d Ed. vol. 1-3, Cold Spring Harbor Press, NY). For example, one method for evaluating the presence, absence, or quantity of the hepsin gene is by Northern blot. Isolated mRNAs from a given biological sample are electrophoresed to separate the mRNA species, and transferred from the gel to a membrane, for example, a nitrocellulose or nylon filter. Labeled hepsin probes are then hybridized to the membrane to identify and quantify the respective mRNAs. The example of amplification based assays include RT-PCR, which is well known in the art (Ausubel et al., Current Protocols in Molecular Biology, eds. 1995 supplement). Quantitative RT-PCR is used preferably to allow the numerical comparison of the level of respective hepsin mRNAs in different samples.

Cancer Diagnosis and Therapies Using Hepsin:

Detection and Measurement of the Hepsin Gene and Protein:

A. Overexpression and Amplification of the Hepsin Gene:

The hepsin gene and its expressed gene product can be used for diagnosis, prognosis, rational drug design, and other therapeutic intervention of tumors and cancers (for example, ovarian cancer, prostate cancer, breast cancer, or lung cancer, etc.).

Detection and measurement of amplification and/or overexpression of the hepsin gene in a biological sample taken from a patient indicates that the patient may have developed a tumor. Particularly, the presence of amplified hepsin DNA leads to a diagnosis of cancer, for example, ovarian cancer, prostate cancer, breast cancer, or lung cancer, etc., with high probability of accuracy. The present invention therefore provides, in one aspect, methods for diagnosing a cancer or tumor in a mammalian tissue by measuring the levels of hepsin mRNA expression in samples taken from the tissue of suspicion, and determining whether hepsin is overexpressed in the tissue. The various techniques, including hybridization based and amplification based methods, for measuring and evaluating mRNA levels are provided herein as discussed supra. The present invention also provides, in another aspect, methods for diagnosing a cancer or tumor in a mammalian tissue by measuring the numbers of hepsin DNA copy in samples taken from the tissue of suspicion, and determining whether the hepsin gene is amplified in the tissue. The various techniques, including hybridization based and amplification based methods, for measuring and evaluating DNA copy numbers are provided herein as discussed supra. The present invention thus provides methods for detecting amplified genes at DNA level and increased expression at RNA level, wherein both the results are indicative of tumor progression.

B. Detection of the Hepsin Protein:

According to the present invention, the detection of increased hepsin protein level in a biological subject may also suggest the presence of a precancerous or cancerous condition in the tissue source of the sample. Protein detection for tumor and cancer diagnostics and prognostics can be carried out by immunoassays, for example, using antibodies directed against a target gene, for example, hepsin. Any methods that are known in the art for protein detection and quantitation can be used in the methods of this invention, including, inter alia, electrophoresis, capillary electrophoresis, high performance liquid chromatography (HPLC), thin layer chromatography (TLC), hyperdiffusion chromatography, immunoelectrophoresis, radioimmunoassay (RIA), enzyme-linked immunosorbent assays (ELISAs), immuno flouorescent assays, Western Blot, etc. Protein from the tissue or cell type to be analyzed may be isolated using standard techniques, for example, as described in Harlow and Lane, Antibodies: A Laboratory Manual (Cold Spring Harbor Laboratory press, Cold Spring Harbor, N.Y. 1988).

The antibodies (or fragments thereof) useful in the present invention can, additionally, be employed histologically, as in immunofluorescence or immunoelectron microscopy, for in situ detection of target gene peptides. In situ detection can be accomplished by removing a histological specimen from a patient, and applying thereto a labeled antibody of the present invention. The antibody (or its fragment) is preferably applied by overlaying the labeled antibody (or fragment) onto a biological sample. Through the use of such a procedure, it is possible to determine not only the presence of the target gene product, for example, hepsin protein, but also their distribution in the examined tissue. Using the present invention, a skilled artisan will readily perceive that any of a wide variety of histological methods (for example, staining procedures) can be modified to achieve such in situ detection.

The biological sample that is subjected to protein detection can be brought in contact with and immobilized on a solid phase support or carrier, for example, nitrocellulose, or other solid support which is capable of immobilizing cells, cell particles, or soluble proteins. The support can then be washed with suitable buffers followed by treatment with the detectably labeled fingerprint gene specific antibody. The solid phase support can then be washed with the buffer a second time to remove unbound antibody. The amount of bound label on the solid support can then be detected by conventional means.

A target gene product-specific antibody, for example, a hepsin antibody can be detectably labeled, in one aspect, by linking the same to an enzyme, for example, horseradish peroxidase, alkaline phosphatase, or glucoamylase, and using it in an enzyme immunoassay (EIA) (see, for example, Voller, A., 1978, The Enzyme Linked Immunosorbent Assay (ELISA), Diagnostic Horizons, 2:1-7; Voller et al., 1978, J. Clin. Pathol., 31:507-520; Butler, J. E., 1981, Meth. Enzymol., 73:482-523; Maggio, E. (ed.), 1980, Enzyme Immunoassay, CRC Press, Boca Raton, Fla.; and Ishikawa et al. (eds), 1981, Enzyme Immunoassay, Kgaku Shoin, Tokyo.) The enzyme bound to the antibody reacts with an appropriate substrate, preferably a chromogenic substrate, in such a manner as to produce a chemical moiety that can be detected, for example, by spectrophotometric or fluorimetric means, or by visual inspection.

In a related aspect, therefore, the present invention provides the use of hepsin antibodies in cancer diagnosis and intervention. Antibodies that specifically bind to hepsin protein and polypeptides can be produced by a variety of methods. Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Such antibodies can be used, for example, in the detection of the target gene, hepsin, or its fingerprint or pathway genes involved in a particular biological pathway, which may be of physiological or pathological importance. These potential pathways or fingerprint genes, for example, may interact with protease activity of hepsin and be involved in tumorigenesis. The hepsin antibodies can also be used in a method for the inhibition of hepsin activity, respectively. Thus, such antibodies can be used in treating tumors and cancers (for example, ovarian cancer, prostate cancer, breast cancer, or lung cancer, etc.); they may also be used in diagnostic procedures whereby patients are tested for abnormal levels of hepsin protein, and/or fingerprint or pathway gene protein associated with hepsin, and for the presence of abnormal forms of such protein.

To produce antibodies to hepsin protein, a host animal is immunized with the protein, or a portion thereof. Such host animals can include, but are not limited to, rabbits, mice, and rats. Various adjuvants can be used to increase the immunological response, depending on the host species, including but not limited to Freund's (complete and incomplete), mineral gels, for example, aluminum hydroxide, surface active substances, for example, lysolecithin, pluronic polyols, polyanions, peptides, oil emulsions, keyhole limpet hemocyanin (KLH), dinitrophenol (DNP), and potentially useful human adjuvants, for example, BCG (Bacille Calmette-Guerin) and Corynebacterium parvum.

Monoclonal antibodies, which are homogeneous populations of antibodies to a particular antigen, for example, hepsin as in the present invention, can be obtained by any technique which provides for the production of antibody molecules by continuous cell lines in culture. These include, but are not limited to the hybridoma technique of Kohler and Milstein, (Nature, 256:495-497, 1975; and U.S. Pat. No. 4,376,110), the human B-cell hybridoma technique (Kosbor et al., Immunology Today, 4:72, 1983; Cole et al., Proc. Natl. Acad. Sci. U.S.A., 80:2026-2030, 1983), and the BV-hybridoma technique (Cole et al., Monoclonal Antibodies And Cancer Therapy (Alan R. Liss, Inc. 1985), pp. 77-96. Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

In addition, techniques developed for the production of “chimeric antibodies” can be made by splicing the genes from a mouse antibody molecule of appropriate antigen specificity together with genes from a human antibody molecule of appropriate biological activity (see, Morrison et al., Proc. Natl. Acad. Sci. USA, 81:6851-6855, 1984; Neuberger et al., Nature, 312:604-608, 1984; Takeda et al., Nature, 314:452-454, 1985; and U.S. Pat. No. 4,816,567). A chimeric antibody is a molecule in which different portions are derived from different animal species, for example, those having a variable region derived from a murine mAb and a container region derived from human immunoglobulin.

Alternatively, techniques described for the production of single chain antibodies (for example, U.S. Pat. No. 4,946,778; Bird, Science, 242:423-426, 1988; Huston et al., Proc. Natl. Acad. Sci. U.S.A., 85:5879-5883, 1988; and Ward et al., Nature, 334:544-546, 1989), and for making humanized monoclonal antibodies (U.S. Pat. No. 5,225,539), can be used to produce anti-differentially expressed or anti-pathway gene product antibodies.

Antibody fragments that recognize specific epitopes can be generated by known techniques. For example, such fragments include but are not limited to: the F(ab′)₂ fragments that can be produced by pepsin digestion of the antibody molecule, and the Fab fragments that can be generated by reducing the disulfide bridges of the F(ab′)₂ fragments. Alternatively, Fab expression libraries can be constructed (Huse et al., Science, 246:1275-1281, 1989) to allow rapid and easy identification of monoclonal Fab fragments with the desired specificity.

C. Use of Hepsin Modulators in Cancer Diagnostics:

Aside from antibodies, the present invention provides, in another aspect, the diagnostic and therapeutic utilities of other molecules and compounds that interact with hepsin protein. Specifically, such compounds can include, but are not limited to, proteins or peptides, for example, soluble peptides, for example, Ig-tailed fusion peptides, comprising extracellular portions of transmembrane proteins of the target, if they exist, and members of random peptide libraries (see, for example, Lam et al., Nature, 354:82-84, 1991; Houghton et al., Nature, 354:84-86, 1991), made of D- and/or L-configuration amino acids, phosphopeptides (including, but not limited to, members of random or partially degenerate phosphopeptide libraries; see, for example, Songyang et al., Cell, 72:767-778, 1993), and small organic or inorganic molecules. In this aspect, the present invention provides a number of methods and procedures to assay or identify compounds that bind to target, i.e., hepsin protein, or to any cellular protein that may interact with the target, and compounds that may interfere with the interaction of the target with other cellular proteins.

In vitro assay systems are provided that are capable of identifying compounds that specifically bind to the target gene product, for example, hepsin protein. The assays all involve the preparation of a reaction mixture of the target gene product, for example, hepsin protein and a test compound under conditions and for a time sufficient to allow the two components to interact and bind, thus forming a complex that can be removed and/or detected in the reaction mixture. These assays can be conducted in a variety of ways. For example, one method involves anchoring the target protein or the test substance to a solid phase, and detecting target protein-test compound complexes anchored to the solid phase at the end of the reaction. In one aspect of such a method, the target protein can be anchored onto a solid surface, and the test compound, which is not anchored, can be labeled, either directly or indirectly. In practice, microtiter plates can be used as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

To conduct the assay, the non-immobilized component is added to the coated surface containing the anchored component. After the reaction is complete, unreacted components are removed, for example, by washing, and complexes anchored on the solid surface are detected. Where the previously immobilized component is pre-labeled, the detection of label immobilized on the surface indicates that complexes were formed. Where the previously non-immobilized component is not pre-labeled, an indirect label can be used to detect complexes anchored on the surface; for example, using a labeled antibody specific for the immobilized component (the antibody, in turn, can be directly labeled or indirectly labeled with a labeled anti-Ig antibody). Alternatively, the reaction can be conducted in a liquid phase, the reaction products separated from unreacted components, and complexes detected, for example, using an immobilized antibody specific for a target gene or the test compound to anchor any complexes formed in solution, and a labeled antibody specific for the other component of the possible complex to detect anchored complexes.

Assays are also provided for identifying any cellular protein that may interact with the target protein, i.e., hepsin protein. Any method suitable for detecting protein-protein interactions can be used to identify novel interactions between target protein and cellular or extracellular proteins. Those cellular or extracellular proteins may be involved in certain cancers, for example, ovarian cancer, prostate cancer, breast cancer, or lung cancer, etc., and represent certain tumorigenic pathways including the target, for example, hepsin. They may thus be denoted as pathway genes.

Methods, for example, co-immunoprecipitation and co-purification through gradients or chromatographic columns, can be used to identify protein-protein interactions engaged by the target protein. The amino acid sequence of the target protein, i.e., hepsin protein or a portion thereof (see SWISS-PROT record P05981, serine protease hepsin), is useful in identifying the pathway gene products or other proteins that interact with hepsin protein. The amino acid sequence can be derived from the nucleotide sequence, or from published database records (SWISS-PROT, PIR, EMBL); it can also be ascertained using techniques well known to a skilled artisan, for example, the Edman degradation technique (see, for example, Creighton, Proteins: Structures and Molecular Principles, 1983, W. H. Freeman & Co., N.Y., 34-49). The nucleotide subsequences of the target gene, for example, hepsin, can be used in a reaction mixture to screen for pathway gene sequences. Screening can be accomplished, for example, by standard hybridization or PCR techniques. Techniques for the generation of oligonucleotide mixtures and the screening are well known (see, for example, Ausubel, supra, and Innis et al. (eds.), PCR Protocols: A Guide to Methods and Applications, 1990, Academic Press, Inc., New York).

By way of example, the yeast two-hybrid system which is often used in detecting protein interactions in vivo is discussed herein. Chien et al. has reported the use of a version of the yeast two-hybrid system (Proc. Natl. Acad. Sci. USA, 1991, 88:9578-9582); it is commercially available from Clontech (Palo Alto, Calif.). Briefly, utilizing such a system, plasmids are constructed that encode two hybrid proteins: the first hybrid protein comprises the DNA-binding domain of a transcription factor, for example, activation protein, fused to a known protein, in this case, a protein known to be involved in a tumor or cancer, and the second hybrid protein comprises the transcription factor's activation domain fused to an unknown protein that is encoded by a cDNA which has been recombined into this plasmid as part of a cDNA library. The plasmids are transformed into a strain of the yeast Saccharomyces cerevisiae that contains a reporter gene, for example, lacZ, whose expression is regulated by the transcription factor's binding site. Either hybrid protein alone cannot activate transcription of the reporter gene. The DNA binding hybrid protein cannot activate transcription because it does not provide the activation domain function, and the activation domain hybrid protein cannot activate transcription because it lacks the domain required for binding to its target site, i.e., it cannot localize to the transcription activator protein's binding site. Interaction between the DNA binding hybrid protein and the library encoded protein reconstitutes the functional transcription factor and results in expression of the reporter gene, which is detected by an assay for the reporter gene product.

The two-hybrid system or similar methods can be used to screen activation domain libraries for proteins that interact with a known “bait” gene product. The hepsin gene product, involved in a number of tumors and cancers, is such a bait according to the present invention. Total genomic or cDNA sequences are fused to the DNA encoding an activation domain. This library and a plasmid encoding a hybrid of the bait gene product, i.e., hepsin protein or polypeptides, fused to the DNA-binding domain are co-transformed into a yeast reporter strain, and the resulting transformants are screened for those that express the reporter gene. For example, the bait gene hepsin can be cloned into a vector such that it is translationally fused to the DNA encoding the DNA-binding domain of the GAL4 protein. The colonies are purified and the (library) plasmids responsible for reporter gene expression are isolated. The inserts in the plasmids are sequenced to identify the proteins encoded by the cDNA or genomic DNA.

A cDNA library of a cell or tissue source that expresses proteins predicted to interact with the bait gene product, for example, hepsin, can be made using methods routinely practiced in the art. According to the particular system described herein, the library is generated by inserting the cDNA fragments into a vector such that they are translationally fused to the activation domain of GAL4. This library can be cotransformed along with the bait gene-GAL4 fusion plasmid into a yeast strain which contains a lacZ gene whose expression is controlled by a promoter which contains a GAL4 activation sequence. A cDNA encoded protein, fused to GAL4 activation domain, that interacts with the bait gene product will reconstitute an active GAL4 transcription factor and thereby drive expression of the lacZ gene. Colonies that express lacZ can be detected by their blue color in the presence of X-gal. cDNA containing plasmids from such a blue colony can then be purified and used to produce and isolate the hepsin-interacting protein using techniques routinely practiced in the art.

In another aspect, the present invention also provides assays for compounds that interfere with gene and cellular protein interactions involving the target hepsin. The target gene product, for example, hepsin protein, may interact in vivo with one or more cellular or extracellular macromolecules, for example, proteins and nucleic acid molecules. Such cellular and extracellular macromolecules are referred to as “binding partners.” Compounds that disrupt such interactions can be used to regulate the activity of the target gene product, for example, hepsin protein, especially mutant target gene product. Such compounds can include, but are not limited to, molecules, for example, antibodies, peptides and other chemical compounds.

The assay systems all involve the preparation of a reaction mixture containing the target gene product hepsin protein, and the binding partner under conditions and for a time sufficient to allow the two products to interact and bind, thus forming a complex. To test a compound for inhibitory activity, the reaction mixture is prepared in the presence and absence of the test compound. The test compound can be initially included in the reaction mixture, or can be added at a time subsequent to the addition of a target gene product and its cellular or extracellular binding partner. Control reaction mixtures are incubated without the test compound or with a placebo. The formation of complexes between the target gene product hepsin protein and the cellular or extracellular binding partner is then detected. The formation of a complex in the control reaction, but not in the reaction mixture containing the test compound, indicates that the compound interferes with the interaction of the target gene product hepsin protein and the interactive binding partner. Additionally, complex formation within reaction mixtures containing the test compound and normal target gene product can be compared to complex formation within reaction mixtures containing the test compound and mutant target gene product. This comparison can be important in the situation where it is desirable to identify compounds that disrupt interactions of mutant but not normal target gene product.

The assays can be conducted in a heterogeneous or homogeneous format. Heterogeneous assays involve anchoring either the target gene product hepsin protein or the binding partner to a solid phase and detecting complexes anchored to the solid phase at the end of the reaction, as described above. In homogeneous assays, the entire reaction is carried out in a liquid phase, as described below. In either approach, the order of addition of reactants can be varied to obtain different information about the compounds being tested. For example, test compounds that interfere with the interaction between the target gene product hepsin protein and the binding partners, for example, by competition, can be identified by conducting the reaction in the presence of the test substance; i.e., by adding the test substance to the reaction mixture prior to or simultaneously with the target gene product hepsin protein and interactive cellular or extracellular binding partner. Alternatively, test compounds that disrupt preformed complexes, for example, compounds with higher binding constants that displace one of the components from the complex, can be tested by adding the test compound to the reaction mixture after complexes have been formed.

In a homogeneous assay, a preformed complex of the target gene product and the interactive cellular or extracellular binding partner product is prepared in which either the target gene products or their binding partners are labeled, but the signal generated by the label is quenched due to complex formation (see, for example, Rubenstein, U.S. Pat. No. 4,109,496). The addition of a test substance that competes with and displaces one of the species from the preformed complex will result in the generation of a signal above background. The test substances that disrupt the interaction between the target gene product hepsin protein and cellular or extracellular binding partners can thus be identified.

In one aspect, the target gene product hepsin protein can be prepared for immobilization using recombinant DNA techniques. For example, the target hepsin coding region can be fused to a glutathione-S-transferase (GST) gene using a fusion vector, for example, pGEX-5X-1, in such a manner that its binding activity is maintained in the resulting fusion product. The interactive cellular or extracellular binding partner product is purified and used to raise a monoclonal antibody, using methods routinely practiced in the art. This antibody can be labeled with the radioactive isotope ¹²⁵I, for example, by methods routinely practiced in the art.

In a heterogeneous assay, the GST-Target gene fusion product is anchored, for example, to glutathione-agarose beads. The interactive cellular or extracellular binding partner is then added in the presence or absence of the test compound in a manner that allows interaction and binding to occur. At the end of the reaction period, unbound material is washed away, and the labeled monoclonal antibody can be added to the system and allowed to bind to the complexed components. The interaction between the target gene product hepsin protein and the interactive cellular or extracellular binding partner is detected by measuring the corresponding amount of radioactivity that remains associated with the glutathione-agarose beads. A successful inhibition of the interaction by the test compound will result in a decrease in measured radioactivity. Alternatively, the GST-target gene fusion product and the interactive cellular or extracellular binding partner can be mixed together in liquid in the absence of the solid glutathione-agarose beads. The test compound is added either during or after the binding partners are allowed to interact. This mixture is then added to the glutathione-agarose beads and unbound material is washed away. Again, the extent of inhibition of the binding partner interaction can be detected by adding the labeled antibody and measuring the radioactivity associated with the beads.

In other aspects of the invention, these same techniques are employed using peptide fragments that correspond to the binding domains of the target gene product, for example, hepsin protein and the interactive cellular or extracellular binding partner (where the binding partner is a product), in place of one or both of the full-length products. Any number of methods routinely practiced in the art can be used to identify and isolate the protein 's binding site. These methods include, but are not limited to, mutagenesis of one of the genes encoding one of the products and screening for disruption of binding in a co-immunoprecipitation assay.

Additionally, compensating mutations in the gene encoding the second species in the complex can be selected. Sequence analysis of the genes encoding the respective products will reveal mutations that correspond to the region of the product involved in interactive binding. Alternatively, one product can be anchored to a solid surface using methods described above, and allowed to interact with and bind to its labeled binding partner, which has been treated with a proteolytic enzyme, for example, trypsin. After washing, a short, labeled peptide comprising the binding domain can remain associated with the solid material, which can be isolated and identified by amino acid sequencing. Also, once the gene coding for the cellular or extracellular binding partner product is obtained, short gene segments can be engineered to express peptide fragments of the product, which can then be tested for binding activity and purified or synthesized.

D. Methods for Cancer Treatment Using Hepsin Modulator:

In another aspect, the present invention provides methods for treating or controlling a cancer or tumor and the symptoms associated therewith. Any of the binding compounds, for example, those identified in the aforementioned assay systems, can be tested for the ability to prevent and/or ameliorate symptoms of tumors and cancers (for example, ovarian cancer, prostate cancer, breast cancer, or lung cancer, etc.). As used herein, inhibit, control, ameliorate, prevent, treat, and suppress collectively and interchangeably mean stopping or slowing cancer formation, development, or growth and eliminating or reducing cancer symptoms. Cell-based and animal model-based trial systems for evaluating the ability of the tested compounds to prevent and/or ameliorate tumors and cancers symptoms are used according to the present invention.

For example, cell based systems can be exposed to a compound suspected of ameliorating ovarian tumor or cancer symptoms, at a sufficient concentration and for a time sufficient to elicit such an amelioration in the exposed cells. After exposure, the cells are examined to determine whether one or more tumor or cancer phenotypes has been altered to resemble a more normal or more wild-type, non-cancerous phenotype. Further, the levels of hepsin mRNA expression and DNA amplification within these cells may be determined, according to the methods provided supra. A decrease in the observed level of expression and amplification would indicate to a certain extent the successful intervention of tumors and cancers (for example, ovarian cancer, prostate cancer, breast cancer, or lung cancer, etc.).

In addition, animal models can be used to identify compounds for use as drugs and pharmaceuticals that are capable of treating or suppressing symptoms of tumors and cancers. For example, animal models can be exposed to a test compound at a sufficient concentration and for a time sufficient to elicit such an amelioration in the exposed animals. The response of the animals to the exposure can be monitored by assessing the reversal of symptoms associated with the tumor or cancer, or by evaluating the changes in DNA copy number and levels of mRNA expression of the target gene, for example, hepsin. Any treatments which reverse any symptom of tumors and cancers, and/or which reduce overexpression and amplification of the target hepsin gene may be considered as candidates for therapy in humans. Dosages of test agents can be determined by deriving dose-response curves.

Moreover, fingerprint patterns or gene, protein expression profiles can be characterized for known cell states, for example, normal or known pre-neoplastic, neoplastic, or metastatic states, within the cell- and/or animal-based model systems. Subsequently, these known fingerprint patterns can be compared to ascertain the ability of a test compound to modify such fingerprint patterns, and to cause the pattern to more closely resemble that of a normal fingerprint pattern. For example, administration of a compound which interacts with and affects hepsin gene expression and amplification may cause the fingerprint pattern of a precancerous or cancerous model system to more closely resemble a control, normal system; such a compound thus will have therapeutic utilities in treating the cancer. In other situations, administration of a compound may cause the fingerprint pattern of a control system to begin to mimic tumors and cancers (for example, ovarian cancer, prostate cancer, breast cancer, or lung cancer, etc.); such a compound therefore acts as a tumorigenic agent, which in turn can serve as a target for therapeutic interventions of the cancer and its diagnosis.

E. Methods for Monitoring Efficacy of Cancer Treatment:

In a further aspect, the present invention provides methods for monitoring the efficacy of a therapeutic treatment regimen of cancer and methods for monitoring the efficacy of a compound in clinical trials for inhibition of tumors. The monitoring can be accomplished by detecting and measuring, in the biological samples taken from a patient at various time points during the course of the application of a treatment regimen for treating a cancer or a clinical trial, the changed levels of expression or amplification of the target gene, for example, hepsin. A level of expression and/or amplification that is lower in samples taken at the later time of the treatment or trial then those at the earlier date indicates that the treatment regimen is effective to control the cancer in the patient, or the compound is effective in inhibiting the tumor. The time course studies should be so designed that sufficient time is allowed for the treatment regimen or the compound to exert its effect.

Therefore, the influence of compounds on tumors and cancers can be monitored both in a clinical trial and in a basic drug screening. In a clinical trial, for example, tumor cells can be isolated from ovarian tumors removed by surgery, and RNA prepared and analyzed by Northern blot analysis or TaqMan RT-PCR as described herein, or alternatively by measuring the amount of protein produced. The fingerprint expression profiles thus generated can serve as putative biomarkers for ovarian or prostate tumors or cancers. Particularly, the expression of hepsin serves as one such biomarker. Thus, by monitoring the level of expression of the differentially or over-expressed genes, for example, hepsin, an effective treatment protocol can be developed using suitable chemotherapeutic anticancer drugs.

F. Use of Modulators to Hepsin Nucleotides in Cancer Treatment:

In another further aspect of this invention, additional compounds and methods for treatment of tumors are provided. Symptoms of tumors and cancers can be controlled by, for example, target gene modulation, and/or by a depletion of the precancerous or cancerous cells. Target gene modulation can be of a negative or positive nature, depending on whether the target resembles a gene (for example, tumorigenic) or a tumor suppressor gene (for example, tumor suppressive). That is, inhibition, i.e., a negative modulation, of an oncogene-like target gene or stimulation, i.e., a positive modulation, of a tumor suppressor-like target gene will control or ameliorate the tumor or cancer in which the target gene is involved. More precisely, “negative modulation” refers to a reduction in the level and/or activity of target gene or its product, for example, hepsin, relative to the level and/or activity of the target gene product in the absence of the modulatory treatment. “Positive modulation” refers to an increase in the level and/or activity of target gene product, for example, hepsin, relative to the level and/or activity of target gene or its product in the absence of modulatory treatment. Particularly because hepsin shares many features with well known oncogenes as discussed supra, inhibition of the hepsin gene, its protein, or its activities will control or ameliorate precancerous or cancerous conditions, for example, ovarian cancer, prostate cancer, breast cancer, or lung cancer, etc.

The techniques to inhibit or suppress a target gene, for example, hepsin that is involved in cancers, i.e., the negative modulatory techniques are provided in the present invention. For example, compounds that exhibit negative modulatory activity on hepsin can be used in accordance with the invention to prevent and/or ameliorate symptoms of tumors and cancers (for example, ovarian cancer, prostate cancer, breast cancer, or lung cancer, etc.). Such molecules can include, but are not limited to, peptides, phosphopeptides, small molecules (molecular weight below about 500), large molecules (molecular weight above about 500), or antibodies (including, for example, polyclonal, monoclonal, humanized, anti-idiotypic, chimeric or single chain antibodies, and Fab, F(ab′)₂ and Fab expression library fragments, and epitope-binding fragments thereof), and nucleic acid molecules that interfere with replication, transcription, or translation of the hepsin gene (for example, antisense nucleic acid molecules, siRNAs and ribozymes).

Antisense, siRNAs and ribozyme molecules that inhibit expression of a target gene, for example, hepsin may reduce the level of the functional activities of the target gene and its product, for example, reduce the catalytic potency of hepsin respectively. Triple helix forming molecules, also related, can be used in reducing the level of target gene activity. These molecules can be designed to reduce or inhibit either wild type, or if appropriate, mutant target gene activity.

For example, anti-sense RNA and DNA molecules act to directly block the translation of mRNA by hybridizing to targeted mRNA and preventing protein translation. With respect to antisense DNA, oligodeoxyribonucleotides derived from the translation initiation site, for example, between the −10 and +10 regions of the target gene nucleotide sequence of interest, are preferred.

Ribozymes are enzymatic RNA molecules capable of catalyzing the specific cleavage of RNA. A review is provided in Rossi, Current Biology, 4:469-471 (1994). The mechanism of ribozyme action involves sequence-specific hybridization of the ribozyme molecule to complementary target RNA, followed by an endonucleolytic cleavage. A composition of ribozyme molecules must include one or more sequences complementary to the target gene mRNA, and must include a well-known catalytic sequence responsible for mRNA cleavage (U.S. Pat. No. 5,093,246). Engineered hammerhead motif ribozyme molecules that may specifically and efficiently catalyze internal cleavage of RNA sequences encoding target protein, for example, hepsin may be used according to this invention in cancer intervention.

Specific ribozyme cleavage sites within any potential RNA target are initially identified by scanning the molecule of interest, for example, hepsin RNA, for ribozyme cleavage sites which include the following sequences, GUA, GUU and GUC. Once identified, short RNA sequences of between 15 and 20 ribonucleotides corresponding to the region of the target gene, for example, hepsin containing the cleavage site can be evaluated for predicted structural features, for example, secondary structure, that can render an oligonucleotide sequence unsuitable. The suitability of candidate sequences can also be evaluated by testing their accessibility to hybridization with complementary oligonucleotides, using ribonuclease protection assays.

The hepsin gene sequences also can be employed in an RNA interference context. The phenomenon of RNA interference is described and discussed in Bass, Nature 411: 428-29 (2001); Elbahir et al., Nature 411: 494-98 (2001); and Fire et al., Nature 391: 806-11 (1998), where methods of making interfering RNA also are discussed. The double-stranded RNA based upon the sequence disclosed herein (for example, GenBank Accession No. M18930 for hepsin) is less than 100 base pairs (“bps”) in length and constituency and preferably is about 30 bps or shorter, and can be made by approaches known in the art, including the use of complementary DNA strands or synthetic approaches. The RNAs that are capable of causing interference can be referred to as small interfering RNAs (“siRNA”), and can cause post-transcriptional silencing of specific genes in cells, for example, mammalian cells (including human cells) and in the body, for example, mammalian bodies (including humans). Exemplary siRNAs according to the invention could have up to 29 bps, 25 bps, 22 bps, 21 bps, 20 bps, 15 bps, 10 bps, 5 bps or any number thereabout or therebetween.

Nucleic acid molecules that can associate together in a triple-stranded conformation (triple helix) and that thereby can be used to inhibit transcription of a target gene, should be single helices composed of deoxynucleotides. The base composition of these oligonucleotides must be designed to promote triple helix formation via Hoogsteen base pairing rules, which generally require sizeable stretches of either purines or pyrimidines on one strand of a duplex. Nucleotide sequences can be pyrimidine-based, which will result in TAT and CGC triplets across the three associated strands of the resulting triple helix. The pyrimidine-rich molecules provide bases complementary to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines on one strand of a duplex.

In instances wherein the antisense, ribozyme, siRNA, and triple helix molecules described herein are used to reduce or inhibit mutant gene expression, it is possible that they can also effectively reduce or inhibit the transcription (for example, using a triple helix) and/or translation (for example, using antisense, ribozyme molecules) of mRNA produced by the normal target gene allele. These situations are pertinent to tumor suppressor genes whose normal levels in the cell or tissue need to be maintained while a mutant is being inhibited. To do this, nucleic acid molecules which are resistant to inhibition by any antisense, ribozyme or triple helix molecules used, and which encode and express target gene polypeptides that exhibit normal target gene activity, can be introduced into cells via gene therapy methods. Alternatively, when the target gene encodes an extracellular protein, it may be preferable to co-administer normal target gene protein into the cell or tissue to maintain the requisite level of cellular or tissue target gene activity. By contrast, in the case of oncogene-like target genes, for example, hepsin, it is the respective normal wild type hepsin gene and its protein that need to be suppressed. Thus, any mutant or variants that are defective in hepsin function or that interferes or completely abolishes its normal function would be desirable for cancer treatment. Therefore, the same methodologies described above to safeguard normal gene alleles may be used in the present invention to safeguard the mutants of the target gene in the application of antisense, ribozyme, and triple helix treatment.

Anti-sense RNA and DNA, ribozyme, and triple helix molecules of the invention can be prepared by standard methods known in the art for the synthesis of DNA and RNA molecules. These include techniques for chemically synthesizing oligodeoxyribonucleotides and oligoribonucleotides well known in the art, for example, for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which also include suitable RNA polymerase promoters, for example, the T7 or Sp6 polymerase promoters. Alternatively, antisense cDNA constructs that synthesize antisense RNA constitutively or inducibly, depending on the promoter used, can be introduced stably into cell lines. Various well-known modifications to the DNA molecules can be introduced as a means for increasing intracellular stability and half-life. Possible modifications include, but are not limited to, the addition of flanking sequences of ribo- or deoxy-nucleotides to the 5′ and/or 3′ ends of the molecule, or the use of phosphorothioate or 2′ O-methyl rather than phosphodiesterase linkages within the oligodeoxyribonucleotide backbone.

In this aspect, the present invention also provides negative modulatory techniques using antibodies. Antibodies can be generated which are both specific for a target gene product and which reduce target gene product activity; they can be administered when negative modulatory techniques are appropriate for the treatment of tumors and cancers, for example, in the case of hepsin antibodies for ovarian cancer treatment.

In instances where the target gene protein to which the antibody is directed is intracellular, and whole antibodies are used, internalizing antibodies are preferred. However, lipofectin or liposomes can be used to deliver the antibody, or a fragment of the Fab region which binds to the target gene epitope, into cells. Where fragments of an antibody are used, the smallest inhibitory fragment which specifically binds to the binding domain of the protein is preferred. For example, peptides having an amino acid sequence corresponding to the domain of the variable region of the antibody that specifically binds to the target gene protein can be used. Such peptides can be synthesized chemically or produced by recombinant DNA technology using methods well known in the art (for example, see Creighton, 1983, supra; and Sambrook et al., 1989, supra). Alternatively, single chain neutralizing antibodies that bind to intracellular target gene product epitopes also can be administered. Such single chain antibodies can be administered, for example, by expressing nucleotide sequences encoding single-chain antibodies within the target cell population by using, for example, techniques, for example, those described in Marasco et al., Proc. Natl. Acad. Sci. U.S.A., 90:7889-7893 (1993). When the target gene protein is extracellular, or is a transmembrane protein, any of the administration techniques known in the art which are appropriate for peptide administration can be used to effectively administer inhibitory target gene antibodies to their site of action. The methods of administration and pharmaceutical preparations are discussed below.

G. Pharmaceutical Applications of Compounds:

The identified compounds that inhibit the expression, synthesis, and/or activity of the target gene, for example, hepsin can be administered to a patient at therapeutically effective doses to prevent, treat, or control a tumor or cancer. A therapeutically effective dose refers to an amount of the compound that is sufficient to result in a measurable reduction or elimination of cancer or its symptoms.

Toxicity and therapeutic efficacy of such compounds can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀ /ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from the cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the method of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

Pharmaceutical compositions for use in the present invention can be formulated by standard techniques using one or more physiologically acceptable carriers or excipients. The compounds and their physiologically acceptable salts and solvates can be formulated and administered orally, intraorally, rectally, parenterally, epicutaneously, topically, transdermally, subcutaneously, intramuscularly, intranasally, sublingually, intradurally, intraocularly, intrarespiratorally, intravenously, intraperitoneally, intrathecal, mucosally, by oral inhalation, nasal inhalation, or rectal administration, for example.

For oral administration, the pharmaceutical compositions can take the form of tablets or capsules prepared by conventional means with pharmaceutically acceptable excipients, for example, binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. The tablets can be coated by methods well known in the art. Liquid preparations for oral administration can take the form of solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoate or sorbic acid. The preparations can also contain buffer salts, flavoring, coloring, and/or sweetening agents as appropriate. Preparations for oral administration can be suitably formulated to give controlled release of the active compound.

For administration by inhalation, the compounds are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use. The compounds can also be formulated in rectal compositions, for example, suppositories or retention enemas, for example, containing conventional suppository bases, for example, cocoa butter or other glycerides.

Furthermore, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can for example comprise metal or plastic foil, for example, a blister pack. The pack or dispenser device can be accompanied by instructions for administration.

The invention is further described by the following examples, which do not limit the invention in any manner.

### EXAMPLES

### Example I

Amplification of the Hepsin DNA in Tumors and Tumor Cell Lines:

The present inventors used DNA microarray-based CGH to survey the genome for gene amplification, and discovered that the hepsin gene is frequently amplified in tumor tissue and cell lines.

The genomic DNAs were isolated from ovarian cancer, prostate cancer, breast cancer, and lung cancer cell lines. They were subjected, along with the same hepsin TaqMan probe set as described supra representing the target, and a reference probe representing a normal non-amplified, single copy region in the genome, to analysis by TaqMan 7700 Sequence Detector following the manufacturer's protocol. Out of 29 ovarian cancer cell lines tested, five were observed to have at least a 2.5 fold increase in their hepsin DNA copies, which gives rise to an amplification frequency of 5/29, i.e., 17% (see Tables 2 and 4). Eight ovarian tumor cell lines were also measured for Hepsin DNA copies, three of which showed at least 2.5 fold increase in their DNA copies, which gives rise to an amplification frequency of 3/8, i.e., 38% (see Tables 2 and 4).

Table 4 shows the DNA copy numbers of the hepsin gene in primary tumors of lung, breast, and prostate. Hepsin gene was not amplified in the tested prostate tumor samples. Hepsin gene was found amplified with a frequency of 3% in the tested lung tumors and a frequency of 6% in the tested breast tumors.

Only samples with the hepsin gene copy number greater than or equal to 2.5 fold are deemed to have been amplified, because of the instrumental detection limit. That is, for example, a Taqman 7700 instrument can not easily distinguish one copy from a two-fold increase in gene copies. However, an increase in hepsin gene copy number less than 2.5 fold can still be considered as an amplification of the gene.

TaqMan epicenter data for hepsin: Referring to FIG. 1, the indicated cell lines or primary tumors were examined for DNA copy number of genes and markers near hepsin to map the boundaries of the amplified regions. Hepsin was found at the epicenter.

### Example II

Overexpression of the Hepsin Gene in Overian Tumors:

Reverse transcriptase (RT)-directed quantitative PCR was performed using the TaqMan 7700 Sequence Detector (Applied Biosystems) to determine the hepsin mRNA level in each sample. Human beta-actin mRNA was used as control. The nucleotide sequences of the hepsin TaqMan probe set used for the detection of mRNA levels detection were:

Hepsin-QF, CACTCAGCCCCGAGACCA;

Hepsin-QR, AGTCCCAGACAGCAGAACAATATTT; and

Hepsin-Qp, [6-FAM]-CCAACCTCACCCTCCTGACCCCC-[TAMRA].

The measurements of the mRNA level of each tumor sample were normalized to the corresponding NAT sample. Relative numeric values of the mRNA levels are shown in Table 1. Of the 5 ovarian cancer cell lines tested, 4 exhibited hepsin overexpression in the range of 10 to 100 fold in the tumor tissue (see Table 1).

### Example III

Overexpression of the Hepsin Gene in Tumors and Tumor Cell Lines:

The frequent overexpression of ovarian hepsin gene is also illustrated in Table 2. Total RNA was isolated from tumors and tumor cell lines using the Trizol reagent. Reverse RT-PCR was performed on the TaqMan 7700 Sequence Dectector, using the same TaqMan probe sets described above. The number of copies of hepsin DNA was also determined, as described below. The measurements of the mRNA level of each tumor sample were normalized to the corresponding NAT sample. Relative numeric values of the mRNA levels are shown in Table 2. Human beta-actin mRNA was used as control. Out of the 29 ovarian tumors tested, 25 expressed hepsin mRNA at a level that is at least five fold greater than that in the normal ovarian tissue, which gives rise to an overexpression frequency of 25/29, i.e., over 86% (see Table 2). In addition, nine ovarian tumor cell lines were analyzed for hepsin expression, five of which expressed hepsin mRNA at a level that is at least five fold greater than that in normal ovarian tissue, which give rise to an overexpression frequency of 5/9, i.e., over 55% (see Table 2).

### Example IV

Overexpression of the Hepsin Gene in prostate Tumors:

Quantitative RT-PCR experiment was performed on the TaqMan 7700 Sequence Detector using the hepsin TaqMan probe set as described above in Example II. The mRNA level of hepsin in each sample was determined, with human beta actin as the reference. The measurements of the mRNA level of each tumor sample were normalized to the corresponding NAT sample. Relative numeric values of the mRNA levels are shown in Table 3. Quantitative RT-PCR analysis with Taqman probes showed that hepsin was found overexpressed in over 70% in prostate tumor samples (10/14 samples, see Table 3). All eight metastatic prostate tumors overexpressed hepsin mRNA, in the range of 7.7 to 89 fold in the tumor tissue.

### Example V

Physical Map of the Amplicon Containing the Hepsin Gene Locus:

The present inventors further demonstrated that hepsin is located at the epicenter of the amplification regions (FIG. 1). FIG. 1 shows the epicenter mapping of 19q13 amplicon which includes hepsin locus. The number of DNA copies for each sample is plotted on the Y-axis, and the X-axis corresponds to nucleotide position based on Human Genome project working draft sequence (http://genome.ucsc.edu/goldenpath/aug2001Tracks.html).

The hepsin gene is indicated by an arrow. Three human genomic DNA clones are presented, i.e., AC020907.4, AC020910.5, and AC024682.3 (not to the scale of actual clone sizes). The genetic markers used were from the following sources: HE07, bases 2602-3583 of genomic DNA clone AC008747.5; HE04, bases 101304-102120 of genomic DNA clone AC022143.6; HE05, bases 1569-3929 of genomic DNA clone AC020907.4, FXYD, bases 50513-50703 of AC024682.3; Hepsin, 3′ UTR of the hepsin gene (bases 70971-71270 of genomic DNA clone AC024682.3); HE12, the coding sequence of hepsin (bases 71834-71978 of genomic DNA clone AC024682.3); HE10A, bases 168971-170218 of genomic DNA clone AC024682.3; HE06, bases 203461-207003 of genomic DNA clone AC020907.4; HE11, bases 1-1912 of genomic DNA clone AC002390.1. CHTN380, 531, 577, 564 and 272, primary ovarian tumors; CAOV1 and CAOV3, ovarian tumor cell lines; LU-12, primary lung tumor; and BR4 and BR26, primary breast tumors. Primary colon and ovarian tumors were obtained from Linda Rodgers and Mike Wigler at the Cold Spring Harbor Laboratory. Primary lung and breast tumors were provided by Jeff Marks at Duke University.

To determine the DNA copy number for each of the gene, corresponding probes to each marker were designed using PrimerExpress 1.0 (Applied Biosystems) and synthesized by Operon Technologies. Subsequently, the target probe (representing the marker), a reference probe (representing a normal non-amplified, single copy region in the genome), and tumor genomic DNA (10 ng) were subjected to analysis by the Applied Biosystems 7700 TaqMan Sequence Detector following the manufacturer's protocol. The number of DNA copies for each sample was plotted against the corresponding marker in FIG. 1. Only one full-length gene hepsin was at the epicenter.

### Example VI

Differential Sensitivity of Ovarian Cancer Cells to Hepsin Antibodies:

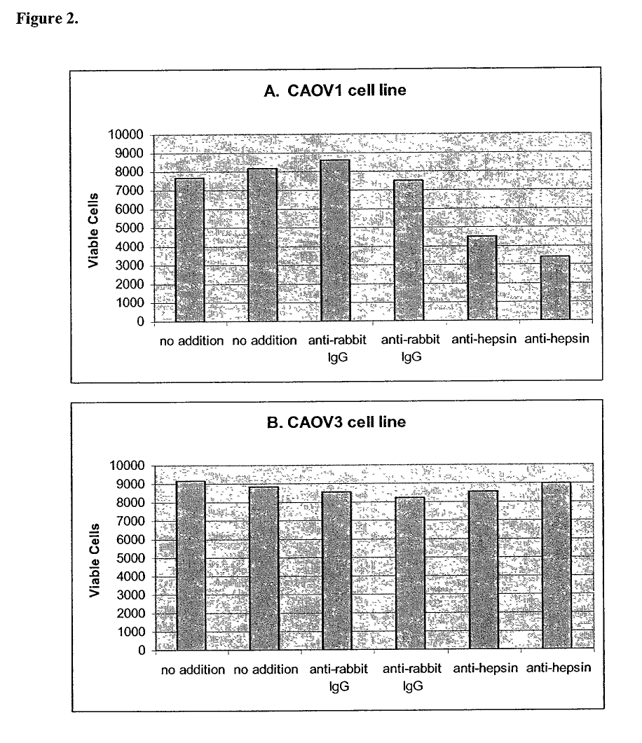

Polyclonal hepsin antibodies were generated using a 19-mer C-terminal peptide (WIFQAIKTHSEASGMVTQL) and affinity purified by Antibody Solutions (Palo Alto, Calif.). Commercial anti-rabbit IgG (control) was purchased from pierce and washed with phosphate buffered saline using microcon spin columns to remove preservatives. The experiments were conducted in duplicate. Two human ovarian cancer cell strains, CAOV1 and CAOV3, were plated out 12-16 hours prior to the 1st dosing of antibodies at 10 μg/mL. Subsequently three additional doses of 10 μg/mL were added to the culture at approximately every 24 hours. The number of viable cells was scored by cell counting with a hemacytomer. The hepsin mRNA expression levels in CAOV1 and CAOV3 were determined by quantitative PCR and were 9.6 and 39, respectively. Although CAOV1 and CAOV3 overexpress hepsin mRNA, the cell lines responded differently to hepsin antibodies (see FIG. 2). CAOV1 was sensitive (see FIG. 2, panel A) and CAOV3 was insensitive (see FIG. 2, panel B) to hepsin antibodies. Therefore, hepsin antibodies can confer death to hepsin-expressing cells of certain genetic makeup.

All above cited references, patents and patent applications are hereby incorporated by reference.

We claim:

1. A method for diagnosing a cancer in a mammal, comprising:
detecting and measuring the hepsin gene copy number in a biological subject from a region of the mammal that is suspected to be precancerous or cancerous, thereby generating data for a test gene copy number; and
comparing the test gene copy number to data for a control gene copy number, wherein an amplification of the gene in the biological subject relative to the control indicates the presence of a precancerous lesion or a cancer in the mammal.

2. The method according to claim 1, wherein the biological subject is selected from the group consisting of ovarian tissue, prostate tissue, breast tissue, and lung tissue.

3. The method according to claim 1, wherein the data is stored in an electronic or a paper format, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, shared server; wherein the data is displayed, transmitted or analyzed via physical transfer, electronic transmission, video display, or telecommunication; wherein the data is compared and compiled at the site of sampling specimens or at a location where the data is transmitted.

4. A method for inhibiting cancer or precancerous growth in a mammalian tissue, comprising contacting the tissue with a nucleotide molecule that interacts with hepsin DNA or RNA and thereby inhibits hepsin gene function.

5. The method according to claim 4, wherein the nucleotide molecule is an antisense nucleotide.

6. The method according to claim 4, wherein the nucleotide molecule is a ribozyme.

7. The method according to claim 4, wherein the nucleotide molecule forms a triple helix with a hepsin-encoding nucleic acid.

8. The method according to claim 4, wherein the tissue is selected from the group consisting of ovarian tissue, prostate tissue, breast tissue, and lung tissue.

9. A method for monitoring the efficacy of a therapeutic treatment regimen in a patient, comprising:
measuring the hepsin gene copy number in a first sample of precancerous or cancer cells obtained from a patient;
administering the treatment regimen to the patient;
measuring the hepsin gene copy number in a second sample of precancerous or cancer cells from the patient at a time following administration of the treatment regimen; and
comparing the gene copy number in the first and the second samples, wherein data showing a decrease in the gene copy number levels in the second sample relative to the first sample indicates that the treatment regimen is effective in the patient.

10. The method according to claim 9, wherein the precancerous or cancer cells are obtained from ovarian tissue, prostate tissue, breast tissue, and lung tissue.

11. The method according to claim 9, wherein the data from measuring or comparing the expression levels is stored in an electronic or a paper format, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, shared server; wherein the data is displayed, transmitted or analyzed via physical transfer, electronic transmission, video display, or telecommunication; wherein the data is compared and compiled at the site of sampling specimens or at a location where the data is transmitted.

12. A method for diagnosing a cancer in a mammal, comprising:
measuring the level of hepsin mRNA transcripts in a biological subject from a region of the mammal that is suspected to be precancerous or cancerous, thereby generating data for a test level; and
comparing the test level to data for a control level, wherein an elevated test level of the biological subject relative to the control level indicates the presence of a cancer or precancerous lesion in the mammal.

13. The method according to claim 12 wherein the biological subject is selected from the group consisting of ovarian tissue, prostate tissue, breast tissue, and lung tissue.

14. The method according to claim 12, wherein the data is stored in an electronic or a paper format, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, shared server; wherein the data is displayed, transmitted or analyzed via physical transfer, electronic transmission, video display, or telecommunication; wherein the data is compared and compiled at the site of sampling specimens or at a location where the data is transmitted.

15. A method for inhibiting cancer or precancerous growth in a mammalian tissue, comprising contacting the tissue with an inhibitor of hepsin protein or a fragment thereof.

16. The method according to claim 15, wherein the cancer or precancerous growth is metastasis.

17. The method according to claim 15, wherein the inhibitor is an antibody that binds to hepsin protein.

18. The method according to claim 15, wherein the inhibitor is an antagonist to hepsin protein.

19. The method according to claim 15, wherein the inhibitor is an antagonist to the protease activity of hepsin protein.

20. The method according to claim 15, wherein the inhibitor is a small molecule.

21. The method according to 15, wherein the tissue is selected from the group consisting of ovarian tissue, prostate tissue, breast tissue, and lung tissue.

22. A method for monitoring the efficacy of a therapeutic treatment regimen in a patient, comprising:
measuring at least one of hepsin mRNA or hepsin expression levels in a first sample of precancerous or cancer cells obtained from a patient;
administering the treatment regimen to the patient;
measuring at least one of hepsin mRNA or hepsin expression levels in a second sample of precancerous or cancer cells from the patient at a time following administration of the treatment regimen; and
comparing at least one of hepsin mRNA or hepsin expression levels in the first and the second samples, wherein data showing a decrease in the levels in the second sample relative to the first sample indicates that the treatment regimen is effective in the patient.

23. The method according to claim 22, wherein the precancerous or cancer cells are obtained ovarian tissue, prostate tissue, breast tissue, and lung tissue.

24. The method according to claim 22, wherein the data from measuring or comparing the expression levels is stored in an electronic or a paper format, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, shared server; wherein the data is displayed, transmitted or analyzed via physical transfer, electronic transmission, video display, or telecommunication; wherein the data is compared and compiled at the site of sampling specimens or at a location where the data is transmitted.

25. An isolated hepsin gene amplicon, wherein the amplicon comprises more than one copy of a polynucleotide selected from the group consisting of:
(a) a polynucleotide encoding the polypeptide set forth in SEQ ID NO: 2;
(b) a polynucleotide set forth in SEQ ID NO: 1;
(c) a polynucleotide having at least about 90% sequence identity to the polynucleotide of (a) or (b); and
(d) a polynucleotide that is overexpressed in tumor cells having at least about 90% sequence identity to the polynucleotide of (a) or (b).

26. The isolated amplicon of claim 25, which comprises a polynucleotide having at least about 90% sequence identity to SEQ ID NO: 1.

27. The isolated amplicon of claim 25, which comprises a polynucleotide having at least about 90% sequence identity to a polynucleotide encoding the polypeptide as set forth in SEQ ID NO:2.

28. The isolated amplicon of claim 25, which comprises a polynucleotide having at least about 95% sequence identity to a polynucleotide encoding SEQ ID NO:2.

29. The isolated amplicon of claim 25, which comprises a polynucleotide encoding the polypeptide set forth in SEQ ID NO:2.

30. The amplicon of claim 25, wherein the polynucleotide comprises SEQ ID NO: 1.

31. The amplicon of claim 25, wherein the polynucleotide sequence encodes the polypeptide of SEQ ID NO:2.

32. A method of making a pharmaceutical composition comprising:
a) identifying a compound which is a moduletor of hepsin;
b) synthesizing the compound; and
c) optionally mixing the compound with suitable additives.

33. A method for diagnosing a cancer in a mammal, comprising:
detecting hepsin protein expression by contacting a biological subject from a region of the mammal that is suspected to be precancerous or cancerous with anti-hepsin antibody, thereby generating data for a test level; and
comparing the test level to data for a control level, wherein an elevated test level of the biological subject relative to the control level indicates the presence of a cancer or precancerous lesion in the mammal.

34. The method according to claim 33, wherein the biological subject is selected from the group consisting of ovarian tissue, prostate tissue, breast tissue, and lung tissue.

35. The method according to claim 33, wherein the data is stored in an electronic or a paper format, wherein the data is stored in an electronic or a paper format, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, shared server; wherein the data is displayed, transmitted or analyzed via physical transfer, electronic transmission, video display, or telecommunication; wherein the data is compared and compiled at the site of sampling specimens or at a location where the data is transmitted.

36. A method of modulating hepsin activities by contacting a biological subject from a region that is suspected to be precancerous or cancerous with a modulator of the hepsin protein.

37. A method according to claim 36 wherein the modulator is a small molecule.

38. A method according to claim 36, wherein said modulator partially or completely inhibits transcription of hepsin.
